(19) United States
(12) Patent Application Publication
(10) Pub. No.: US 2025/0325974 A1
(43) Pub. Date: Oct. 23, 2025

(54) ANION EXCHANGE CHROMATOGRAPHIC PARTICLES WITH CONTROLLED GRAFTED POLYMER CHAINS

(71) Applicant: Waters Technologies Corporation, Milford, MA (US)

(72) Inventors: Kristine Joy Camacho, Franklin, MA (US); MingCheng Xu, Lexington, MA (US); Matthew A. Lauber, North Smithfield, RI (US); Szabolcs Fekete, Ville La Grand (FR)

(73) Assignee: Waters Technologies Corporation, Milford, MA (US)

(21) Appl. No.: 18/986,059

(22) Filed: Dec. 18, 2024

Related U.S. Application Data
(60) Provisional application No. 63/611,801, filed on Dec. 19, 2023.

(51) Int. Cl. B01J 41/14; B01D 15/36; B01J 41/05; B01J 47/02

(52) U.S. Cl. CPC B01J 41/14; B01D 15/363; B01J 41/05; B01J 47/02

(57) ABSTRACT

The present disclosure pertains to compositions comprising a non-porous particle core coated with a hydrophilic polymer with surface-grafted polyionic chains. In some aspects, the present disclosure pertains to chromatographic separation devices that comprise such compositions. In some further aspects, the present disclosure pertains to chromatographic methods that comprise: (a) loading a sample onto a chromatographic column comprising such compositions and (b) flowing a mobile phase through the column.

### RELATED APPLICATION

This application claims the benefit of and priority to U.S. Provisional Application No. 63/611,801, filed Dec. 19, 2023, the entire disclosure of which is incorporated herein by reference.

### BACKGROUND

Stationary phase materials for liquid chromatography (LC) are generally classified into two types: organic materials, e.g., polydivinylbenzene, and inorganic materials typified by silica. Many organic materials are chemically stable against strongly alkaline and strongly acidic mobile phases, allowing flexibility in the choice of mobile phase pH. However, organic chromatographic materials generally result in columns with low efficiency, particularly with low molecular-weight analytes. Many organic chromatographic materials not only lack the mechanical strength of typical chromatographic silicas and also shrink and swell when the composition of the mobile phase is changed.

Silica is the material most widely used in High Performance Liquid Chromatography (HPLC). The most common applications employ silica that has been surface-derivatized with an organic functional group such as octadecyl (C₁₈), octyl (C₈), phenyl, amino, cyano, etc. As stationary phases for HPLC, these stationary phase materials result in columns that have high efficiency and do not show evidence of shrinking or swelling.

Anion exchange chromatography (AEX) separates molecules based on the differences in number and localization of negative surface charges of an analyte. An area of further study of AEX includes drug development. For instance, adeno-associated virus (AAV) is becoming a widely used vector for delivering therapeutic genes for many diseases. Full AAV capsids carry negatively charged DNA within their structures and have a slightly lower isoelectric point (pI) than empty AAV capsids (generally a difference in the range of 0.4 pH units). Due to this relative surface charge difference between empty and full capsids, anion exchange chromatography serves as a suitable technique to separate and quantify full and empty AAV capsids (Hejmowski, A. L., et al., Biotechnology Journal, 17: e2100219 (2022). Conventional free radical polymerization methods and surface derivatization techniques are commonly used for the preparation of ion exchange stationary phases. While these methods resulted to the fabrication of a wide range of silica, silica-polymer hybrid, or polymer-based ion exchange stationary phases, they are characterized by poor control over the surface density of the ionic functionalities and the polymer chain lengths for graft-based ion exchange stationary phases.

Therefore, there remains a need for further ion exchange chromatographic particles that provide improved recovery and lower carry-over of large nucleic acids (specifically single stranded ribonucleic acid (RNA) and DNA fragments), batch to batch consistency, and column lifetime and performance.

### SUMMARY

In general, the present disclosure relates to anion exchange chromatographic particles. The anion exchange chromatographic particles of the present disclosure are designed to be particularly useful in the analysis of large nucleic acids (specifically single stranded ribonucleic acid (RNA) and DNA fragments). In certain examples, the particles of the present technology are created by a two-step synthetic process that involves 1) covalent attachment of initiator molecules on the hydrophilic layer of a non-porous polymeric resin and 2) controlled grafting of polymeric chains bearing anion exchange functionalities to and/or from the initiator-modified resin.

In various aspects, the present disclosure pertains to compositions comprising a non-porous particle core coated with a hydrophilic polymer with surface-grafted polyionic chains, wherein the polyionic chains have a structure represented by Formula I, II, or II:

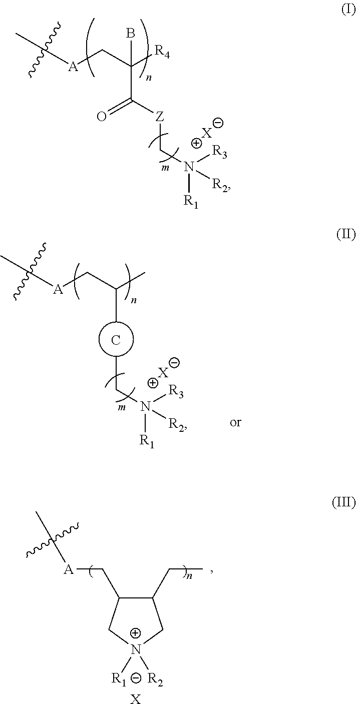

wherein the squiggly line, A, B, C, n, Z, m, R₁, R₂, R₃, R₄ and X are defined herein.

In some examples, the particle core comprises an organic polymer having a polymer backbone that contains C—C covalent bonds, C—O covalent bonds, C—N covalent bonds, O—N covalent bonds, or a combination thereof. In some examples, the particle core comprises an organic polymer having a polymer backbone that contains C—C covalent bonds.

In some examples, the particle core has a particle size ranging from 1 μm to 30 μm.

In some examples, the particle core has a pore volume that is less than 0.1 cc/g.

In some examples, the particle core is coated with an intermediate organic polymer primer layer.

In some examples, the intermediate organic polymer primer layer comprises a hydrophilic polymer copolymerized from the particle surface of a crosslinker, such as ethylene dimethacrylate (EDMA) and a functional monomer bearing an epoxy or hydroxyl group (e.g., glycidyl methacrylate (GMA)). In some examples, the intermediate organic polymer primer layer comprises an EDMA-GMA layer.

In some examples, the hydrophilic coating comprises a hydrophilic polymer layer with either random structures or with multiple domains with different chemistries.

In some examples, including all the previous examples, A is an optionally substituted C₁-C₄ alkyl, C₁-C₄ thioalkyl, or C₁-C₄ alkyl ester group. In some examples, A is

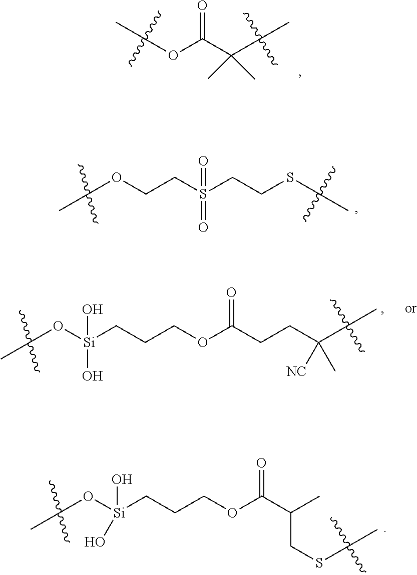

In some examples, including all the previous examples, B is hydrogen, optionally substituted C₁-C₄ alkyl or carbonotrithioate group. In some examples, B is methyl.

In some examples, including all the previous examples, C is phenyl or C₅-C₆ heteroaryl (e.g., imidazolyl and pyridyl). In some further examples, C is C₅-C₆ heteroaryl.

In some examples, including all the previous examples, the polyionic chains are of Formula I or II, and R₁, R₂, and R₃ are each independently H, methyl, ethyl, isopropyl, t-butyl, or benzyl. In some examples, the polyionic chains are of Formula I or II, and one of R₁, R₂, and R₃ is a lone pair.

In some examples, the polyionic chains are of Formula III, and R₁ and R₂ are each independently H, methyl, ethyl, isopropyl, t-butyl, or benzyl. In some examples, the polyionic chains are of Formula III, and R₁ or R₂ is a lone pair.

In some examples, including all the previous examples, the polyionic chains have any one of structures:

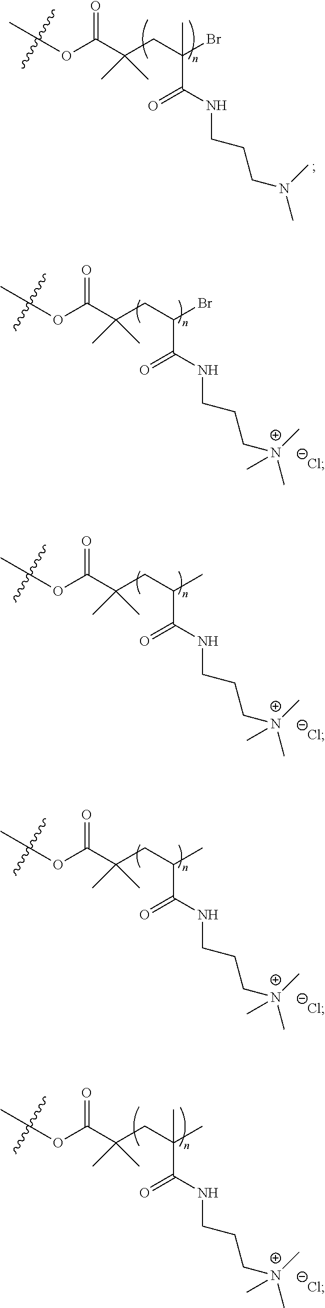

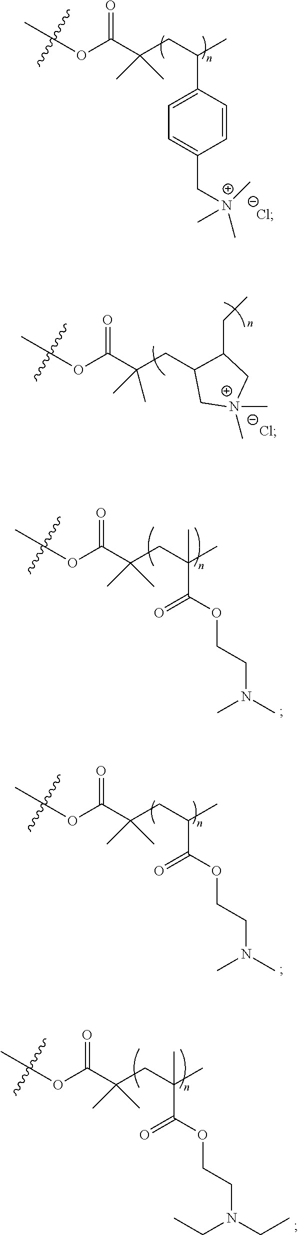

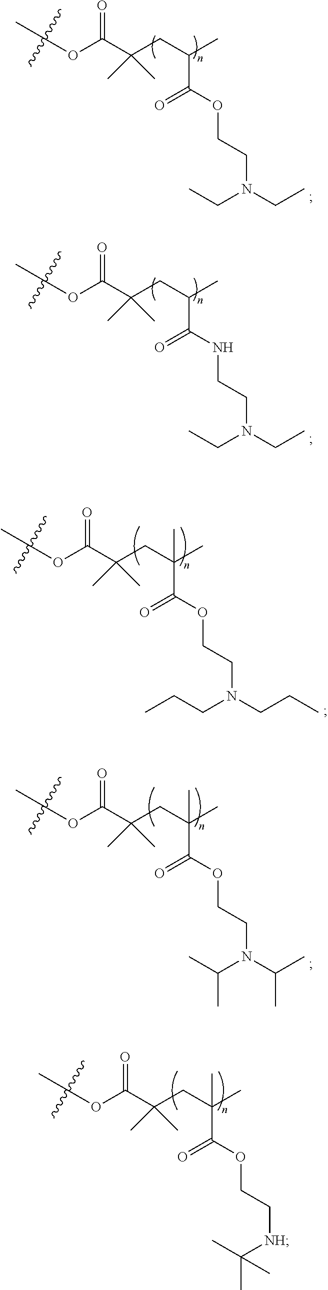

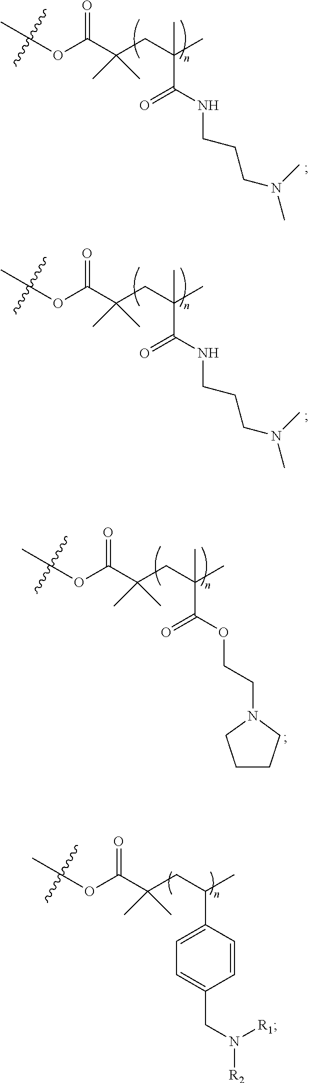

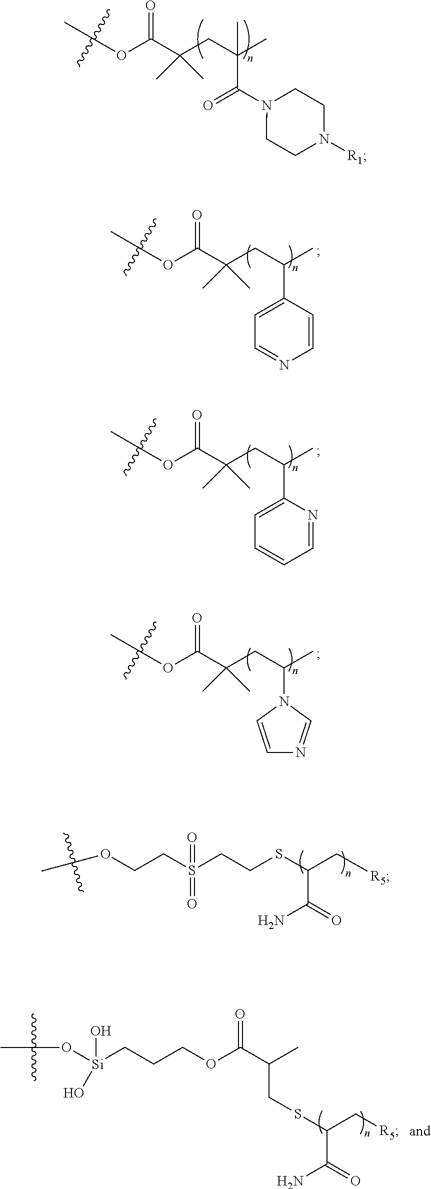

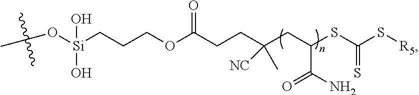

wherein R₅ is optionally substituted C₁-C₄ alkyl.

In some examples, the polyionic chains are strong anion exchangers, weak anion exchangers, or a mixture thereof.

In some examples, including all the previous examples, the polyionic chains form homopolymers, di-block copolymers, di-block mixed mode ion exchangers, random mixed ion exchangers, gradient ion exchangers, multi-component ion exchangers, or bottlebrush ion exchangers.

In some examples, including all the previous examples, the composition has a grafting density between 0.5-15 μmol chains/m².

In some aspects, the present disclosure pertains to a chromatographic separation device that comprises the composition of the present disclosure.

In some further aspects, the present disclosure pertains to a chromatographic method comprising: (a) loading a sample onto a chromatographic column comprising the composition of the present disclosure and (b) flowing a mobile phase through the column.

In some examples, the sample comprises large nucleic acids. In some examples, the sample comprises single stranded ribonucleic acid (RNA) or deoxyribonucleic acid (DNA) fragments.

Precise control over surface functionalities, architecture, and ionic capacities of these anion exchange stationary phases offers greater influence on their selectivity for different applications. The compositions and methods of the present disclosure provide high resolution separations for existing and new modalities for analytical liquid chromatography (LC) and LC/mass spectrometry (MS) as well as lab scale preparations. They also provide improved recovery and lower carry-over of large nucleic acids (specifically single stranded RNA), batch to batch consistency, and column lifetime and performance. Methods for preparing the particles/anion exchange stationary phases are also provided herein.

### BRIEF DESCRIPTION OF THE DRAWINGS

FIG. 1A-FIG. 1E show schematic illustrations of a nonporous particle core coated with a hydrophilic polymer with surface-grafted polyionic chains, a synthetic scheme for making the particle, exemplary strong anion exchange ligands containing quaternary ammonium functionality, and exemplary weak anion exchange ligands with various functionalities and their corresponding pKa values, in accordance with the examples of the present disclosure. FIG. 1A illustrates a generic illustration of the nonporous particle core with surface grafted polyionic chains. FIG. 1B illustrates structural features of exemplary surface-grafted polyionic chains generated via various living polymerization techniques that allow control and variation of polymer compositions and structures. FIG. 1C illustrates a two-step process for the synthesis of polymeric anion exchange particles in accordance with the examples of the present disclosure. FIG. 1D illustrates exemplary strong anion exchange ligands containing quaternary ammonium functionality. FIG. 1E illustrates exemplary weak anion exchange ligands with various functionalities and their corresponding pKa values.

FIG. 2A-FIG. 2B illustrate exemplary surface-grafted polyionic chains. FIG. 2A illustrates an exemplary strong anion exchanger (SAX). FIG. 2B illustrates an exemplary weak anion exchanger (WAX).

FIG. 3A-FIG. 3C illustrate DNA ladder separations using an ion-exchange (IEX) column sold under the tradename PROTEIN-PAK™ Hi Res Q, commercially available from Waters Technologies Corporation, and SAX columns. FIG. 3A illustrates a 1 kb+ DNA ladder. FIG. 3B illustrates DNA fragments separation using PROTEIN-PAK™ Hi Res Q. FIG. 3C illustrates DNA fragments separation using SAX column in accordance with the present technology under optimized gradient conditions.

FIG. 4A-FIG. 4B illustrate DNA ladder separation using a high ionic capacity (IC) SAX column (157 μmol/g) under different methods. FIG. 4A illustrates DNA ladder separation using a unified test method with 650-1000 mM NaCl, pH 7.4. FIG. 4B illustrates DNA ladder separation using an optimized unified test method with 650-1000 mM NaCl, pH 9.0.

FIG. 5A-FIG. 5B illustrate DNA ladder separations using a high IC WAX column (140 μmol/g) under different methods. FIG. 5A illustrates DNA ladder separations using a unified test method with 650-1000 mM NaCl, pH 7.4. FIG. 5B illustrates DNA ladder separations using an optimized unified test method with 650-1000 mM NaCl, pH 9.0.

### DETAILED DESCRIPTION

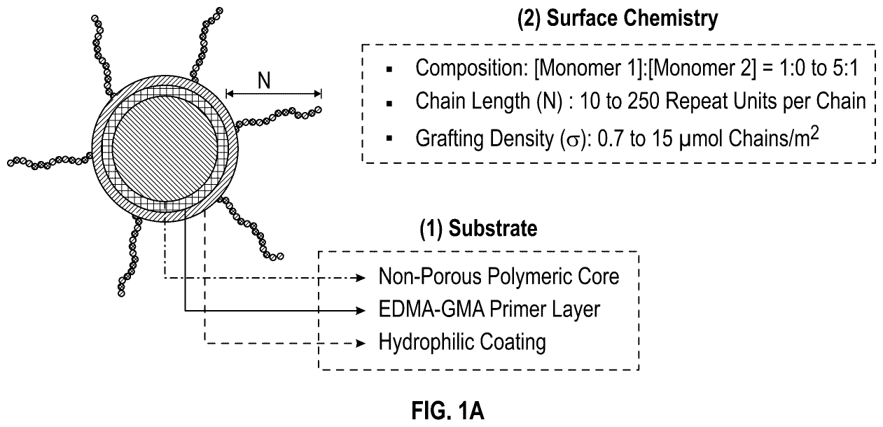

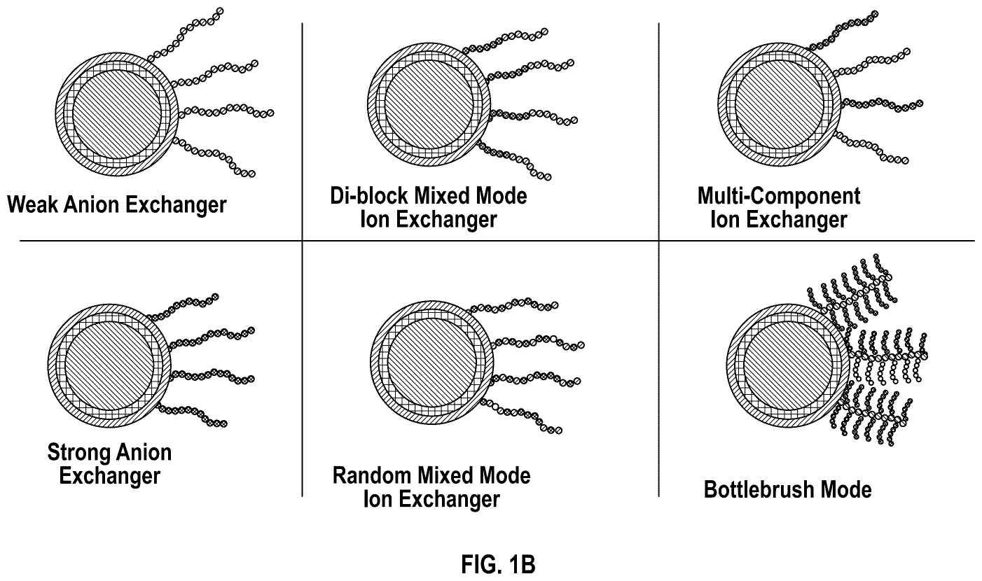

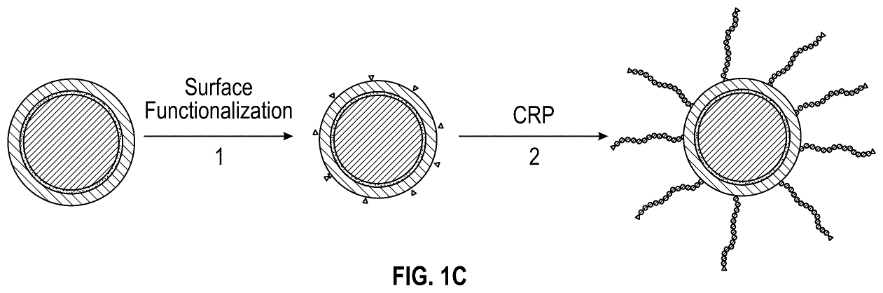

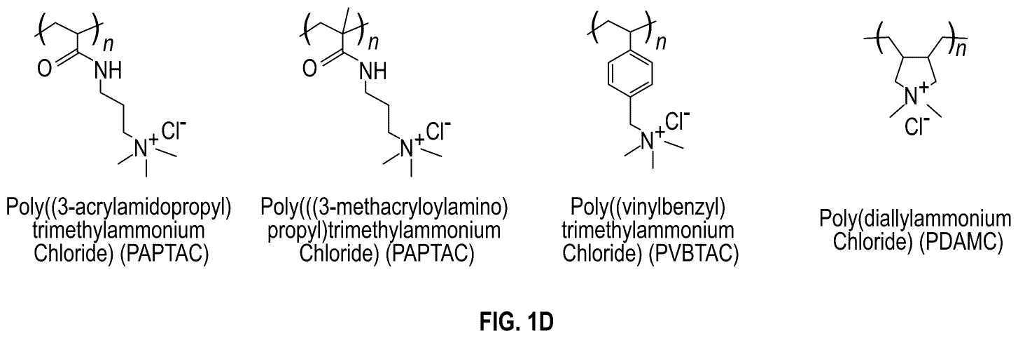

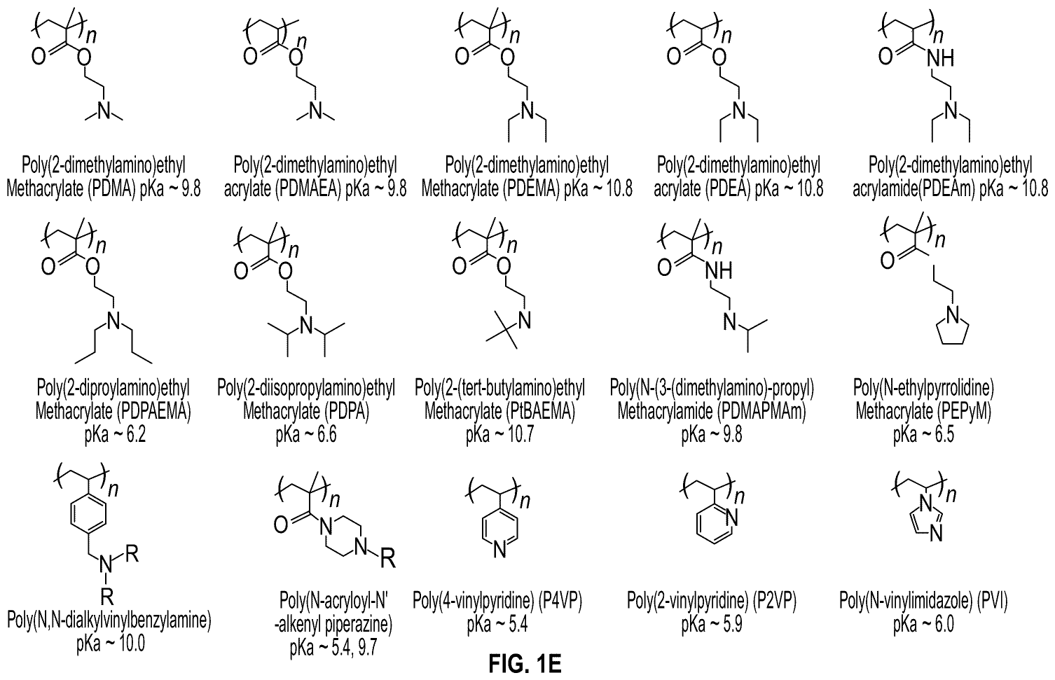

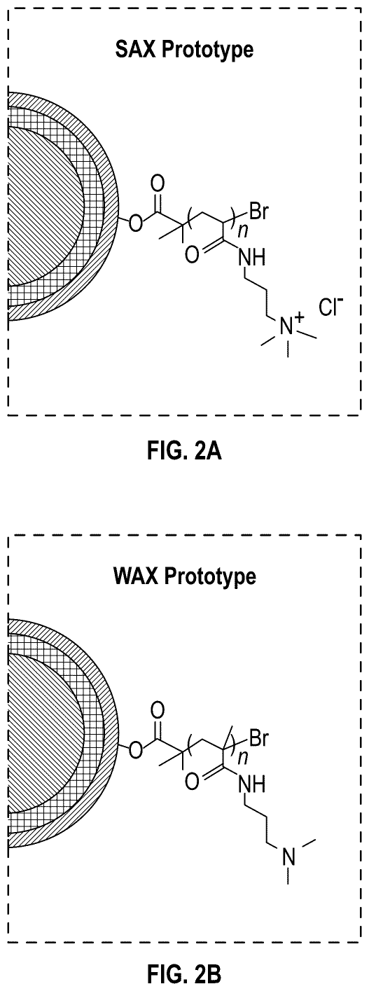

Broadly, the present disclosure is directed to anion exchange chromatographic particles comprising a non-porous particle core coated with a hydrophilic polymer with surface-grafted polyionic chains. More specifically, the anion exchange chromatographic particles comprise a coated core with hydrophilic polymer layer and surface grafted polymer chains bearing anion exchange functionalities, as illustrated in FIG. 1A-FIG. 1B. The anion exchange chromatographic particles are designed to be particularly useful in the analysis of large nucleic acids (specifically single stranded ribonucleic acid (RNA) and DNA fragments). By utilizing controlled polymerization technique during the grafting process, anion exchange particles with high density of ion exchange groups on a narrowly dispersed, long chain, and flexible backbone were achieved. Methods for preparing the particles are also provided below (FIG. 1C).

The anion exchange chromatographic particles and methods of making these particles with tailored surface chains will be described in more detail below after the following definition section.

### Definitions

Unless defined otherwise, all technical and scientific terms used herein have the same meaning as is commonly understood by one of skill in the art to which the subject matter herein belongs. As used in the specification and the appended claims, unless specified to the contrary, the following terms have the meaning indicated in order to facilitate the understanding of the present disclosure.

As used in the description and the appended claims, the singular forms “a,” “an,” and “the” include plural referents unless the context clearly dictates otherwise. Therefore, for example, reference to “a composition” includes mixtures of two or more such compositions, reference to “an inhibitor” includes mixtures of two or more such inhibitors, and the like.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim.

With respect to compositions of the present disclosure, and to the extent the following terms are used herein to further describe them, the following definitions apply.

As used herein, the term “halogen (halo)” refers to a fluorine, chlorine, bromine, or iodine atom.

As used herein, the term “alkyl” refers to a saturated linear or branched-chain monovalent hydrocarbon radical. In some examples, and to the extent not specified otherwise with respect to any one or more groups in the compounds of formula (I), the alkyl radical is a C₁-C₄ group. In other examples, the alkyl radical is a C₀-C₄, C₀-C₃, C₁-C₄, C₁-C₃ or C₁-C₂ group (wherein C₀ alkyl refers to a bond). Representative examples of alkyl groups include methyl, ethyl, 1-propyl, 2-propyl, i-propyl, 1-butyl, 2-methyl-1-propyl, 2-butyl, and 2-methyl-2-propyl. In some examples, an alkyl group is a C₁-C₃ alkyl group. In some examples, an alkyl group is a C₃-C₅ branched-chain alkyl group.

As used herein, the term “alkylene” refers to a straight or branched divalent hydrocarbon chain linking the rest of the molecule to a radical group, consisting solely of carbon and hydrogen. The alkylene chain may be attached to the rest of the molecule through a single bond and to the radical group through a single bond. In some examples, and to the extent not specified otherwise, the alkylene group contains one to 4 carbon atoms (C₁-C₄ alkylene (e.g., methylene, ethylene, propylene, and n-butylene)). In other examples, an alkylene group contains one to 3 carbon atoms (C₁-C₃ alkylene). In other examples, an alkylene group contains one to 2 carbon atoms (C₁-C₂ alkylene). In other examples, an alkylene group contains one carbon atom (C₁ alkylene).

As used herein, the term “alkenyl” refers to a linear or branched-chain monovalent hydrocarbon radical with at least one carbon-carbon double bond. An alkenyl includes radicals having “cis” and “trans” orientations, or alternatively, “E” and “Z” orientations. In some examples, and to the extent not specified otherwise, the alkenyl radical is a C₂-C₆ group. In other examples, the alkenyl radical is a C₂-C₆ or C₂-C₃ group. Examples include ethenyl or vinyl, prop-1-enyl, prop-2-enyl, 2-methylprop-1-enyl, but-1-enyl, but-2-enyl, but-3-enyl, buta-1,3-dienyl, 2-methylbuta-1,3-diene, hex-1-enyl, hex-2-enyl, hex-3-enyl, hex-4-enyl and hexa-1,3-dienyl.

The terms “alkoxyl” or “alkoxy” as used herein refer to an alkyl group, as defined above, having an oxygen radical attached thereto. Representative alkoxyl groups include methoxy, ethoxy, propyloxy, tert-butoxy and the like. An “ether” is two hydrocarbyl groups covalently linked by an oxygen. Accordingly, the substituent of an alkyl that renders that alkyl an ether is or resembles an alkoxyl, such as can be represented by one of —O-alkyl, —O-alkenyl, and —O-alkynyl.

As used herein, the term “alkoxylene” refers to a saturated monovalent aliphatic radical of the general formula (—O—CₙH₂ₙ—) where n represents an integer (e.g., 1, 2, 3, 4, 5, 6, or 7) and is inclusive of both straight-chain and branched-chain radicals. The alkoxylene chain may be attached to the rest of the molecule through a single bond and to the radical group through a single bond. In some examples, and to the extent not specified otherwise, the alkoxylene group contains one to 3 carbon atoms (—O—C₁-C₃ alkoxylene). In other examples, an alkoxylene group contains one to 5 carbon atoms (—O—C₁-C₅ alkoxylene).

As used herein, the term “cyclic group” broadly refers to any group that used alone or as part of a larger moiety, contains a saturated, partially saturated, or aromatic ring system e.g., carbocyclic (cycloalkyl, cycloalkenyl), heterocyclic (heterocycloalkyl, heterocycloalkenyl), aryl and heteroaryl groups. Cyclic groups may have one or more (e.g., fused) ring systems. Therefore, for example, to the extent not specified otherwise, a cyclic group can contain one or more (e.g., 1, 2, or 3) carbocyclic, heterocyclic, aryl or heteroaryl groups.

As used herein, the term “carbocyclic” (also “carbocyclyl”) refers to a group that used alone or as part of a larger moiety, contains a saturated, partially unsaturated, or aromatic ring system having 3 to 20 carbon atoms, that is alone or part of a larger moiety (e.g., an alkcarbocyclic group). The term carbocyclyl includes mono-, bi-, tri-, fused, bridged, and spiro-ring systems, and combinations thereof. To the extent not specified otherwise, in one embodiment, carbocyclyl includes 3 to 15 carbon atoms (C₃-C₁₅). In one embodiment, carbocyclyl includes 3 to 12 carbon atoms (C₃-C₁₂). In another embodiment, carbocyclyl includes C₃-C₈, C₃-C₁₀ or C₅-C₁₀. In another embodiment, carbocyclyl, as a monocycle, includes C₃-C₈, C₃-C₆ or C₅-C₆. In some examples, carbocyclyl, as a bicycle, includes C₇-C₁₂. In another embodiment, carbocyclyl, as a spiro system, includes C₅-C₁₂. Representative examples of monocyclic carbocyclyls include cyclopropyl, cyclobutyl, cyclopentyl, 1-cyclopent-1-enyl, 1-cyclopent-2-enyl, 1-cyclopent-3-enyl, cyclohexyl, perdeuteriocyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl, phenyl, and cyclododecyl; bicyclic carbocyclyls having 7 to 12 ring atoms include [4,3], [4,4], [4,5], [5,5], [5,6] or [6,6] ring systems, such as for example bicyclo[2.2.1]heptane, bicyclo[2.2.2]octane, naphthalene, and bicyclo[3.2.2]nonane. Representative examples of spiro carbocyclyls include spiro[2.2]pentane, spiro[2.3]hexane, spiro[2.4]heptane, spiro[2.5]octane and spiro[4.5]decane. The term carbocyclyl includes aryl ring systems as defined herein. The term carbocycyl also includes cycloalkyl rings (e.g., saturated, or partially unsaturated mono-, bi-, or spiro-carbocycles). The term carbocyclic group also includes a carbocyclic ring fused to one or more (e.g., 1, 2 or 3) different cyclic groups (e.g., aryl or heterocyclic rings), where the radical or point of attachment is on the carbocyclic ring.

As used herein, the term “heterocyclyl” refers to a “carbocyclyl” that used alone or as part of a larger moiety, contains a saturated, partially unsaturated or aromatic ring system, wherein one or more (e.g., 1, 2, 3, or 4) carbon atoms have been replaced with a heteroatom (e.g., O, N, N(O), S, S(O), or S(O)₂). The term heterocyclyl includes mono-, bi-, tri-, fused, bridged, and spiro ring systems, and combinations thereof. In some examples, to the extent not specified otherwise, a heterocyclyl refers to a 3 to 15 membered heterocyclyl ring system. In some examples, a heterocyclyl refers to a 3 to 12 membered heterocyclyl ring system. In some examples, a heterocyclyl refers to a saturated ring system, such as a 3 to 12 membered saturated heterocyclyl ring system. In some examples, a heterocyclyl refers to a heteroaryl ring system, such as a 5 to 14 membered heteroaryl ring system. The term heterocyclyl also includes C₃-C₈ heterocycloalkyl, which is a saturated or partially unsaturated mono-, bi-, or spiro-ring system containing 3-8 carbons and one or more (1, 2, 3 or 4) heteroatoms.

In some examples, a heterocyclyl group includes 3-12 ring atoms and includes monocycles, bicycles, tricycles and spiro ring systems, wherein the ring atoms are carbon, and one to 5 ring atoms is a heteroatom such as nitrogen, sulfur, or oxygen. In some examples, to the extent not specified otherwise, heterocyclyl includes 3- to 7-membered monocycles having one or more heteroatoms selected from nitrogen, sulfur, or oxygen. In some examples, heterocyclyl includes 4- to 6-membered monocycles having one or more heteroatoms selected from nitrogen, sulfur, or oxygen. In some examples, heterocyclyl includes 3-membered monocycles. In some examples, heterocyclyl includes 4-membered monocycles. In some examples, heterocyclyl includes 5-6 membered monocycles. In some examples, the heterocyclyl group includes 0 to 3 double bonds. In any of the foregoing examples, heterocyclyl includes 1, 2, 3 or 4 heteroatoms. Any nitrogen or sulfur heteroatom may optionally be oxidized (e.g., NO, SO, SO₂), and any nitrogen heteroatom may optionally be quaternized (e.g., [NR₄]⁺Cl⁻, [NR₄]⁺OH⁻). Representative examples of heterocyclyls include oxiranyl, aziridinyl, thiiranyl, azetidinyl, oxetanyl, thietanyl, 1,2-dithietanyl, 1,3-dithietanyl, pyrrolidinyl, dihydro-1H-pyrrolyl, dihydrofuranyl, tetrahydropyranyl, dihydrothienyl, tetrahydrothienyl, imidazolidinyl, piperidinyl, piperazinyl, morpholinyl, thiomorpholinyl, 1,1-dioxo-thiomorpholinyl, dihydropyranyl, tetrahydropyranyl, hexahydrothiopyranyl, hexahydropyrimidinyl, oxazinanyl, thiazinanyl, thioxanyl, homopiperazinyl, homopiperidinyl, azepanyl, oxepanyl, thiepanyl, oxazepinyl, oxazepanyl, diazepanyl, 1,4-diazepanyl, diazepinyl, thiazepinyl, thiazepanyl, tetrahydrothiopyranyl, oxazolidinyl, thiazolidinyl, isothiazolidinyl, 1,1-dioxoisothiazolidinonyl, oxazolidinonyl, imidazolidinonyl, 4,5,6,7-tetrahydro[2H]indazolyl, tetrahydrobenzoimidazolyl, 4,5,6,7-tetrahydrobenzo[d]imidazolyl, 1,6-dihydroimidazol[4,5-d]pyrrolo[2,3-b]pyridinyl, thiazinyl, thiophenyl, oxazinyl, thiadiazinyl, oxadiazinyl, dithiazinyl, dioxazinyl, oxathiazinyl, thiatriazinyl, oxatriazinyl, dithiadiazinyl, imidazolinyl, dihydropyrimidyl, tetrahydropyrimidyl, 1-pyrrolinyl, 2-pyrrolinyl, 3-pyrrolinyl, indolinyl, thiapyranyl, 2H-pyranyl, 4H-pyranyl, dioxanyl, 1,3-dioxolanyl, pyrazolinyl, pyrazolidinyl, dithianyl, dithiolanyl, pyrimidinonyl, pyrimidindionyl, pyrimidin-2,4-dionyl, piperazinonyl, piperazindionyl, pyrazolidinylimidazolinyl, 3-azabicyclo[3.1.0]hexanyl, 3,6-diazabicyclo[3.1.1]heptanyl, 6-3-azabicyclo[3.1.1]heptanyl, 3-azabicyclo[4.1.0]heptanyl, azabicyclo[3.1.1]heptanyl, 2-azabicyclo[3.2.1]octanyl, 8-azabicyclo[3.2.1]octanyl, 2-azabicyclo[2.2.2]hexanyl, azabicyclo[2.2.2]octanyl, 8-azabicyclo[2.2.2]octanyl, 7-oxabicyclo[2.2.1]heptane, azaspiro[3.5]nonanyl, azaspiro[2.5]octanyl, azaspiro[4.5]decanyl, 1-azaspiro[4.5]decan-2-only, azaspiro[5.5]undecanyl, tetrahydroindolyl, octahydroindolyl, tetrahydroisoindolyl, tetrahydroindazolyl, 1,1-dioxohexahydrothiopyranyl. Examples of 5-membered heterocyclyls containing a sulfur or oxygen atom and one to three nitrogen atoms are thiazolyl, including thiazol-2-yl and thiazol-2-yl N-oxide, thiadiazolyl, including 1,3,4-thiadiazol-5-yl and 1,2,4-thiadiazol-5-yl, oxazolyl, for example oxazol-2-yl, and oxadiazolyl, such as 1,3,4-oxadiazol-5-yl, and 1,2,4-oxadiazol-5-yl. Example 5-membered ring heterocyclyls containing 2 to 4 nitrogen atoms include imidazolyl, such as imidazol-2-yl; triazolyl, such as 1,3,4-triazol-5-yl; 1,2,3-triazol-5-yl, 1,2,4-triazol-5-yl, and tetrazolyl, such as 1H-tetrazol-5-yl. Representative examples of benzo-fused 5-membered heterocyclyls are benzoxazol-2-yl, benzthiazol-2-yl and benzimidazol-2-yl. Example 6-membered heterocyclyls contain one to three nitrogen atoms and optionally a sulfur or oxygen atom, for example pyridyl, such as pyrid-2-yl, pyrid-3-yl, and pyrid-4-yl; pyrimidyl, such as pyrimid-2-yl and pyrimid-4-yl; triazinyl, such as 1,3,4-triazin-2-yl and 1,3,5-triazin-4-yl; pyridazinyl, in particular pyridazin-3-yl, and pyrazinyl.

Therefore, the term heterocyclic embraces N-heterocyclyl groups which as used herein refer to a heterocyclyl group containing at least one nitrogen and where the point of attachment of the heterocyclyl group to the rest of the molecule is through a nitrogen atom in the heterocyclyl group. To the extent not specified otherwise, representative examples of N-heterocyclyl groups include 1-morpholinyl, 1-piperidinyl, 1-piperazinyl, 1-pyrrolidinyl, pyrazolidinyl, imidazolinyl and imidazolidinyl. The term heterocyclic also embraces C-heterocyclyl groups which as used herein refer to a heterocyclyl group containing at least one heteroatom and where the point of attachment of the heterocyclyl group to the rest of the molecule is through a carbon atom in the heterocyclyl group. To the extent not specified otherwise, representative examples of C-heterocyclyl radicals include 2-morpholinyl, 2- or 3- or 4-piperidinyl, 2-piperazinyl, and 2- or 3-pyrrolidinyl. The term heterocyclic also embraces heterocyclylalkyl groups which as disclosed above refer to a group of the formula —R^{c}-heterocyclyl where R^{c} is an alkylene chain. The term heterocyclic also embraces heterocyclylalkoxy groups which as used herein refer to a radical bonded through an oxygen atom of the formula —O—R^{c}-heterocyclyl where R^{c} is an alkylene chain.

As used herein, the term “aryl” used alone or as part of a larger moiety (e.g., “aralkyl”, wherein the terminal carbon atom on the alkyl group is the point of attachment, e.g., a benzyl group), “aralkoxy” wherein the oxygen atom is the point of attachment, or “aroxyalkyl” wherein the point of attachment is on the aryl group) refers to a group that includes monocyclic, bicyclic or tricyclic, carbon ring system, that includes fused rings, wherein at least one ring in the system is aromatic. In some examples, the aralkoxy group is a benzoxy group. The term “aryl” may be used interchangeably with the term “aryl ring”. In one embodiment, to the extent not specified otherwise, aryl includes groups having 6-18 carbon atoms. In another embodiment, aryl includes groups having 6-10 carbon atoms. Examples of aryl groups include phenyl, naphthyl, anthracyl, biphenyl, phenanthrenyl, naphthacenyl, 1,2,3,4-tetrahydronaphthalenyl, 1H-indenyl, 2,3-dihydro-1H-indenyl, naphthyridinyl, and the like, which may be substituted or independently substituted by one or more substituents described herein. A particular aryl is phenyl. In some examples, to the extent not specified otherwise, an aryl group includes an aryl ring fused to one or more (e.g., 1, 2 or 3) different cyclic groups (e.g., carbocyclic rings or heterocyclic rings), where the radical or point of attachment is on the aryl ring.

Therefore, the term aryl embraces aralkyl groups (e.g., benzyl) which as disclosed above refer to a group of the formula —R^{c}-aryl where R^{c} is an alkylene chain such as methylene or ethylene. In some examples, to the extent not specified otherwise, the aralkyl group is an optionally substituted benzyl group. The term aryl also embraces aralkoxy groups which as used herein refer to a group bonded through an oxygen atom of the formula —O—R^{c}-aryl where R^{c} is an alkylene chain such as methylene or ethylene.

As used herein, the term “heteroaryl” used alone or as part of a larger moiety (e.g., “heteroarylalkyl” (also “heteroaralkyl”), or “heteroarylalkoxy” (also “heteroaralkoxy”), refers to a monocyclic, bicyclic, or tricyclic ring system having 5 to 14 ring atoms, wherein at least one ring is aromatic and contains at least one heteroatom. In one embodiment, to the extent not specified otherwise, heteroaryl includes 5-6 membered monocyclic aromatic groups where one or more ring atoms is nitrogen, sulfur or oxygen that is independently optionally substituted. Representative examples of heteroaryl groups include thienyl, furyl, imidazolyl, pyrazolyl, thiazolyl, isothiazolyl, oxazolyl, isoxazolyl, triazolyl, thiadiazolyl, oxadiazolyl, tetrazolyl, thiatriazolyl, oxatriazolyl, pyridyl, pyrimidyl, imidazopyridyl, pyrazinyl, pyridazinyl, triazinyl, tetrazinyl, tetrazolo[1,5-b]pyridazinyl, purinyl, deazapurinyl, benzoxazolyl, benzofuryl, benzothiazolyl, benzothiadiazolyl, benzotriazolyl, benzoimidazolyl, indolyl, 1,3-thiazol-2-yl, 1,3,4-triazol-5-yl, 1,3-oxazol-2-yl, 1,3,4-oxadiazol-5-yl, 1,2,4-oxadiazol-5-yl, 1,3,4-thiadiazol-5-yl, 1H-tetrazol-5-yl, 1,2,3-triazol-5-yl, and pyrid-2-yl N-oxide. The term “heteroaryl” also includes groups in which a heteroaryl is fused to one or more (e.g., 1, 2 or 3) cyclic (e.g., carbocyclyl, or heterocyclyl) rings, where the radical or point of attachment is on the heteroaryl ring. Nonlimiting examples include indolyl, indolizinyl, isoindolyl, benzothienyl, benzothiophenyl, methylenedioxyphenyl, benzofuranyl, dibenzofuranyl, indazolyl, benzimidazolyl, benzodioxazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl and pyrido[2,3-b]-1,4-oxazin-3 (4H)-one. A heteroaryl group may be mono-, bi- or tri-cyclic. In some examples, a heteroaryl group includes a heteroaryl ring fused to one or more (e.g., 1, 2 or 3) different cyclic groups (e.g., carbocyclic rings or heterocyclic rings), where the radical or point of attachment is on the heteroaryl ring, and in some examples wherein the point of attachment is a heteroatom contained in the heterocyclic ring.

The term heteroaryl also embraces N-heteroaryl groups which as used herein refers to a heteroaryl group, as defined above, and which contains at least one nitrogen atom and where the point of attachment of the N-heteroaryl group to the rest of the molecule is through a nitrogen atom in the heteroaryl group. The term heteroaryl further embraces C-heteroaryl groups which as used herein refer to a heteroaryl group as defined above and where the point of attachment of the heteroaryl group to the rest of the molecule is through a carbon atom in the heteroaryl group. The term heteroaryl further embraces heteroarylalkyl groups which as disclosed above refer to a group of the formula —R^{c}-heteroaryl, wherein R^{c} is an alkylene chain as defined above. The term heteroaryl further embraces heteroaralkoxy (or heteroarylalkoxy) groups which as used herein refer to a group bonded through an oxygen atom of the formula —O—R^{c}-heteroaryl, where R^{c} is an alkylene group as defined above.

To the extent not disclosed otherwise for any particular group(s), representative examples of substituents may therefore include alkyl, substituted alkyl (e.g., C₁-C₆, C₁-C₅, C₁-C₄, C₁-C₃, C₁-C₂, C₁), alkoxy (e.g., C₁-C₆, C₁-C₅, C₁-C₄, C₁-C₃, C₁-C₂, C₁), substituted alkoxy (e.g., C₁-C₆, C₁-C₅, C₁-C₄, C₁-C₃, C₁-C₂, C₁), haloalkyl (e.g., CF3), alkenyl (e.g., C₂-C₆, C₂-C₅, C₂-C₄, C₂-C₃, C₂), substituted alkenyl (e.g., C₂-C₆, C₂-C₅, C₂-C₄, C₂-C₃, C₂), alkynyl (e.g., C₂-C₆, C₂-C₅, C₂-C₄, C₂-C₃, C₂), substituted alkynyl (e.g., C₂-C₆, C₂-C₅, C₂-C₄, C₂-C₃, C₂), cyclic (e.g., C₃-C₁₂, C₅-C₆), substituted cyclic (e.g., C₃-C₁₂, C₅-C₆), carbocyclic (e.g., C₃-C₁₂, C₅-C₆), substituted carbocyclic (e.g., C₃-C₁₂, C₅-C₆), heterocyclic (e.g., C₃-C₁₂, C₅-C₆), substituted heterocyclic (e.g., C₃-C₁₂, C₅-C₆), aryl (e.g., benzyl and phenyl), substituted aryl (e.g., substituted benzyl or phenyl), heteroaryl (e.g., pyridyl or pyrimidyl), substituted heteroaryl (e.g., substituted pyridyl or pyrimidyl), aralkyl (e.g., benzyl), substituted aralkyl (e.g., substituted benzyl), halogen, hydroxyl, aryloxy (e.g., C₆-C₁₂, C₆), substituted aryloxy (e.g., C₆-C₁₂, C₆), alkylthio (e.g., C₁-C₆), substituted alkylthio (e.g., C₁-C₆), arylthio (e.g., C₆-C₁₂, C₆), substituted arylthio (e.g., C₆-C₁₂, C₆), cyano, carbonyl, substituted carbonyl, carboxyl, substituted carboxyl, amino, substituted amino, amido, substituted amido, thio, substituted thio, sulfinyl, substituted sulfinyl, sulfonyl, substituted sulfonyl, sulfinamide, substituted sulfinamide, sulfonamide, substituted sulfonamide, urea, substituted urea, carbamate, substituted carbamate, amino acid, and peptide groups.

The present disclosure pertains to compositions comprising a non-porous particle core coated with a hydrophilic polymer with surface-grafted polyionic chains, and methods of using the compositions. In some examples, the particle core is coated with an intermediate organic polymer primer layer. In some examples, the intermediate layer comprises a hydrophilic polymer copolymerized from the particle surface of a crosslinker, such as ethylene dimethacrylate (EDMA) and a functional monomer bearing an epoxy or hydroxyl group (e.g., glycidyl methacrylate (GMA). In some examples, the hydrophilic coating comprises a hydrophilic polymer layer with either random structures (e.g., crosslinked polyglycidol/glyceroltriglycidyl ether) or with multiple domains with different chemistries (e.g., crosslinked polyglycidol/glyceroltriglycidyl ether and/or polyethylenedimethacrylate/glyceroltriglycidyl ether).

The non-porous particle cores of the present disclosure are typically spherical. The non-porous particle cores of the present disclosure typically range from 1 to 14 microns in diameter, more typically, from 1 to 6 microns in diameter. Particle diameter is measured herein by Coulter Counter ((Beckman Coulter, Multisizer 4e Coulter Counter, Brea, CA, USA) by dispersing a sample in methanol containing 5% lithium chloride. A greater than 70,000 particle count is run using a 30 μm aperture in the volume mode for each sample. Using the Coulter principle, volumes of particles are converted to diameter, where a particle diameter is the equivalent spherical diameter, which is the diameter of a sphere whose volume is identical to that of the particle. Particle diameter measured as the 50% cumulative diameter of the volume-based particle size distribution.

The non-porous particle cores of the present disclosure have good stability, even at pH's greater than 12 and less than 1, in some examples.

The non-porous particle cores of the present disclosure are also typically narrowly dispersed in particle size. As defined herein, a collection of particles is “narrowly dispersed in particle size” when a ratio of 90% cumulative volume diameter divided by the 10% cumulative volume diameter is less than 1.4 when measured by Beckman Coulter, Multisizer 4e Coulter Counter.

The non-porous core particle, the hydrophilic coating, and surface-grafted polyionic chains forming the anion exchange chromatographic particles of the present technology will now be discussed in more detail.

### Non-Porous Particle Core

The non-porous particle cores for use in the present disclosure comprise at least one organic polymer. The non-porous particle cores typically contain more than 95% organic polymer, more typically more than 97.5% organic polymer, even more typically more than 99% organic polymer.

As described, the polymer particle cores are “non-porous,” which is defined herein to mean that the polymer particle cores have a pore volume that is less than 0.1 cc/g. Preferably, organic polymer cores have a pore volume that is less than 0.05 cc/g, and preferably less than 0.02 cc/g, in some examples. Pore volume is determined using methods known in the art based on multipoint nitrogen sorption experiments (Micromeritics ASAP 2400; Micromeritics Instruments Inc., Norcross, GA). In some examples, the particle core has a pore volume that is less than 0.1 cc/g.

The particle cores typically range, for example, from 1 to 30 microns in diameter, more typically, from 1 to 5 microns in diameter. The particle cores are typically narrowly distributed in particle size. In some examples, the particle core has a particle size ranging from 1 μm to 14 μm.

Elementally, polymer particle cores for use in the present disclosure include those that are composed of carbon and hydrogen, those composed of carbon, hydrogen, and oxygen, those composed of carbon, hydrogen, and nitrogen, and those composed of carbon, hydrogen, nitrogen, and oxygen. The backbones of the organic polymer chains forming the particle cores may contain C—C, C—O, C—N and/or O—N covalent bonds. In some examples (e.g., in the case of a polymer formed by radical polymerization of vinyl groups), the backbone of the at least one organic polymer chains may contain only C—C covalent bonds.

As noted above, the non-porous particle cores for use in the present disclosure comprise at least one organic polymer. The at least one organic polymer comprises residues of one or more organic monomers. The one or more organic monomers residues forming the least one organic polymer may be selected from residues of hydrophobic organic monomers, residues of hydrophilic organic monomers, or a mixture of residues of hydrophobic organic monomers and residues of hydrophilic organic monomers.

Hydrophobic organic monomers may be selected, for example, from a C₂-C₁₈ olefin monomer and/or a monomer comprising a C₆-C₁₈ monocyclic or multicyclic carbocyclic group (e.g., a phenyl group, a phenylene group, naphthalene group, etc.). Specific examples of hydrophobic organic monomers include, for example, monofunctional and multifunctional aromatic monomers such as styrene, alkyl substituted styrene, halo substituted styrene, divinylbenzene, and vinylbenzyl chloride, monofunctional and multifunctional olefin monomers such as ethylene, propylene or butylene, monofunctional and multifunctional fluorinated monomers such as fluoroethylene, 1,1-(difluoroethylene), tetrafluoroethylene, chlorotrifluoroethylene, hexafluoropropylene, perfluoropropylvinylether, or perfluoromethylvinylether, monofunctional or multifunctional acrylate monomers having a higher alkyl or carbocyclic groups, for example, monofunctional or multifunctional acrylate monomers having a C₆-C₁₈ alkyl, alkenyl or alkynyl group or a C₆-C₁₈ saturated, unsaturated or aromatic carbocyclic group, monofunctional or multifunctional methacrylate monomers having a higher alkyl or carbocyclic group, for example, monofunctional or multifunctional methacrylate monomers having a C₆-C₁₈ alkyl, alkenyl or alkynyl group or a C₆-C₁₈ saturated, unsaturated or aromatic carbocyclic group, among others.

Hydrophilic organic monomers may be selected, for example, from monofunctional or multifunctional organic monomers having an amide group, monofunctional or multifunctional organic monomers having an ester group, monofunctional or multifunctional organic monomers having a carbonate group, monofunctional or multifunctional organic monomers having a carbamate group, monofunctional or multifunctional organic monomers having a urea group, monofunctional or multifunctional organic monomers having a hydroxyl group, and monofunctional or multifunctional organic monomers having a nitrogen-containing heterocyclic group, among other possibilities. Specific examples of hydrophilic organic monomers include, for example, vinyl pyridine, N-vinylpyrrolidone, N-vinyl-piperidone, N-vinyl caprolactam, lower alkyl acrylates (e.g., methyl acrylate, ethyl acrylate, etc.), lower alkyl methacrylates (e.g., methyl methacrylate, ethyl methacrylate, etc.), vinyl acetate, acrylamide or methacrylamide monomers, hydroxypolyethoxy allyl ether monomers, ethoxy ethyl methacrylate, ethylene glycol dimethacrylate, methylene bisacrylamide, allyl methacrylate, or diallyl maleate.

In various examples, the non-porous particle cores comprise residues of multifunctional hydrophobic organic monomers such as divinylbenzene and/or multifunctional hydrophilic organic monomers, such as ethylene glycol dimethacrylate, methylene bisacrylamide or allyl methacrylate, in order to provide crosslinks in the organic copolymer. In certain examples, DVB 80 may be employed, which is an organic monomer mixture that comprises divinylbenzene (80%) as well as a mixture of ethyl-styrene isomers, diethylbenzene, and can include other isomers as well.

In various examples, the non-porous particle core may comprise residues of only multifunctional organic monomers. In various examples, the polymer may comprise residues of both multifunctional organic monomers and monofunctional organic monomers.

In some examples, the non-porous particle cores are created in which a central region of the cores is formed from an organic copolymer containing multifunctional organic monomer residues and monofunctional organic monomer residues and an outer region of the cores is formed from an organic homopolymer containing only multifunctional organic monomer residues. In some examples, non-porous particle cores are created in which a central region of the cores is formed from an organic copolymer containing multifunctional organic monomer residues and monofunctional organic monomer residues and an outer region of the cores is also formed from an organic copolymer containing multifunctional organic monomer residues and monofunctional organic monomer residues, but wherein a molar ratio of the multifunctional organic monomer residues relative to the monofunctional organic monomer residues is increased in the outer core region relative to the central core region. In a particular example, non-porous particle cores are created in which a central region of the cores is formed from DVB and styrene and an outer region of the cores is formed from DVB. In other embodiment, the molar ratio of the multifunctional organic monomer residues relative to the monofunctional organic monomer residues is decreased in the outer core region relative to the central core region.

In various examples, an entire non-porous particle core may be formed from an organic polymer that comprises residues of multifunctional organic monomers but does not contain resides of monofunctional organic monomers. In various examples, an entire non-porous particle core may be formed from an organic polymer that comprises residues of both multifunctional organic monomers and monofunctional organic monomers.

The polymers forming the non-porous particle cores can be prepared via a number of processes and mechanisms including, but not limited to, chain addition and step condensation processes, radical, anionic, cationic, ring-opening, group transfer, metathesis, and photochemical mechanisms. In particularly beneficial examples, the non-porous particle cores are prepared via free radical polymerization.

The polymer particle cores of the present disclosure can be prepared in some examples by a dispersion polymerization process in which a homogeneous solution is formed, wherein monomers, initiator and stabilizer are combined in a solvent or solvent mixture. As polymerization proceeds, the initially formed polymers precipitate from the homogeneous solution to form nuclei. The nuclei that form still bear reactive sites such as radicals which allow them to keep growing by continuous capture and incorporation of monomers and/or oligomers from the solution.

In an exemplary process based on radical polymerization, one or more solvents and one or more stabilizers are purged with nitrogen to remove dissolved oxygen. Then, one or more monomers, including at least one multifunctional monomer, and a radical polymerization initiator are added. Radical polymerization is initiated by raising the temperature for several hours, typically under agitation. Based on the desired particle diameter, further radical polymerization initiator and further monomer may be added to the reaction mixture to allow the particle further to grow to the desired size. After reaction, the particles may be thoroughly washed and dried under vacuum.

Any radical initiator that is compatible with the organic phase may be used, either alone or in a mixture of such radical initiators. In particular examples, the radical initiators are capable of being heat activated or photoactivated. In specific examples, the radical initiator is a peroxide, a peroxyacetate, a persulfate, an azo initiator, or a mixture thereof.

Where the initiator is a thermal initiator, the resulting solution may then be heated to an elevated temperature under agitation to activate the thermal initiator(s) and maintained at elevated temperature until polymerization is complete. Where the initiator is a photoinitiator, the resulting solution may then be illuminated under agitation with light having a suitable wavelength to activate the photoinitiator(s) and maintained until polymerization is complete. Suitable organic monomers for use in the organic phase are described above.

Solvent systems for the formation of non-porous particle cores include methanol, ethanol, isopropanol, 2-methoxyehanol, water, acetonitrile, p-xylene, and toluene.

Stabilizers that can be employed for the formation of non-porous particle cores include, for example, polyvinylpyrrolidone (PVP), non-ionic surfactants including alkylphenol ethoxylates (e.g., TRITON™ N-57, available from Dow Chemical), polyvinyl alcohol (PVA) such as SELVOL™ Polyvinyl Alcohol solution, available from Sekisui Special Chemicals), modified celluloses, including alkyl-modified celluloses such as methyl celluloses (e.g., METHOCEL™, available from DuPont) and hydrophobically modified celluloses hydroxyethylcellulose stabilizers such as NATROSOL™ cetyl modified hydroxyethylcellulose (available from Ashland), and ionic surfactants including sodium alkyl sulfates such as sodium dodecyl sulfate (SDS) and sodium oleyl sulfate, among others.

A particular embodiment of non-porous particle core formation, in which the monomer is a combination of DVB and styrene, the initiator is 2,2′-Azobis(2-methylpropionitrile) (AIBN), the solvent system is a combination of reagent alcohol and p-xylene, and the stabilizer is polyvinyl pyrrolidone (PVP 40), is schematically illustrated in FIG. 1A.

Once formed, the organic polymer cores may contain surface moieties from which further polymerization can proceed. For example, nonporous polymer particle cores formed from free radical polymerization commonly contain residual radical-polymerizable unsaturated surface moieties (e.g., ethylenyl moieties, vinyl moieties, methacryloxy moieties, or acryloxy moieties, etc.), from which further core growth can proceed. Such further polymerization may be used to increase the size of a given batch of polymer particle cores by adding an additional thickness of non-porous organic polymer to previously formed non-porous polymer particle cores. In some examples, the particle core is coated with an intermediate organic polymer primer layer. In some examples, the intermediate layer comprises a hydrophilic polymer copolymerized from the particle surface of a crosslinker, such as EDMA and a functional monomer bearing an epoxy or hydroxyl group (e.g., GMA and solketal methacrylate). “Functional monomers” as used herein refers to monomers having particularly reactive side-chain groups that may be used in the synthesis of more complex polymers. In some examples, the primer layer comprises an EDMA-GMA layer.

Hydrophilic Polymer with Surface-Grafted Polyionic Chains

In some examples, the non-porous particle cores are coated with a hydrophilic polymer with surface-grafted polyionic chains. In some examples, the hydrophilic coating comprises a hydrophilic polymer layer with either random structures or with multiple domains with different chemistries (e.g., functional groups that may covalently or electrostatically bond to the intermediate the primer layer).

In some examples, the hydrophilic polymer coating can have a thickness ranging from 1 to 300 nm, for example ranging anywhere from 1 to 3 to 10 to 30 to 100 to 300 nm (i.e., ranging between any two of the preceding values).

The hydrophilic polymer coating may comprise, for example, a crosslinked polymer network that is formed on the intermediate polymer layer.

Hydrophilic polymers can be prepared via a number of processes and mechanisms including, but not limited to, chain addition and step condensation processes, radical, anionic, cationic, ring-opening, group transfer, metathesis, and photochemical mechanisms.

The hydrophilic polymers comprise, for example, hydrophilic organic monomers selected from monofunctional or multifunctional organic monomers having an amide group, monofunctional or multifunctional organic monomers having an ester group, monofunctional or multifunctional organic monomers having a carbonate group, monofunctional or multifunctional organic monomers having a carbamate group, monofunctional or multifunctional organic monomers having a urea group, monofunctional or multifunctional organic monomers having a hydroxyl group, and monofunctional or multifunctional organic monomers having a nitrogen-containing heterocyclic group, among other possibilities. Specific examples of hydrophilic organic monomers include, for example, vinyl pyridine, N-vinylpyrrolidone, N-vinyl-piperidone, N-vinyl caprolactam, lower alkyl acrylates (e.g., methyl acrylate, ethyl acrylate, etc.), lower alkyl methacrylates (e.g., methyl methacrylate, ethyl methacrylate, etc.), vinyl acetate, acrylamide or methacrylamide monomers, hydroxypolyethoxy allyl ether monomers, ethoxy ethyl methacrylate, ethylene glycol dimethacrylate, methylene bisacrylamide, allyl methacrylate, or diallyl maleate.

As noted above, surface hydrophilic polymers include those comprising amide monomer residues. Examples of amide monomer residues include amide monomer residues having the formula,

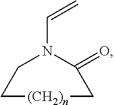

wherein n is an integer from 1-3 (i.e., N-vinyl pyrrolidone, N-vinyl-2-piperidinone or N-vinyl caprolactam). Examples of amide monomer residues also include amide monomer residues having the formula

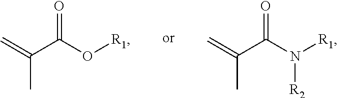

wherein R₁ is selected from C₁-C₆ alkylene, C₂-C₆ alkenylene, C₂-C₆ alkynylene, C₆-C₁₈ arylene groups, and wherein R₂ is selected from H, C₁-C₆ alkylene, C₂-C₆ alkenylene, C₂-C₆ alkynylene, C₆-C₁₈ arylene groups.

As also noted above, surface hydrophilic polymers include those comprising amine monomer residues. Examples of amine monomer residues include aminoalkyl acrylates, aminoalkyl methacrylates, dialkylaminoalkyl acrylates, or dialkylaminoalkyl methacrylates, including amino-C₁-C₄-alkyl acrylates, amino-C₁-C₄-alkyl methacrylates, di-C₁-C₄-alkylamino-C₁-C₄-alkyl acrylates, di-C₁-C₄-alkylamino-C₁-C₄-alkyl methacrylates, such as 2-aminoethyl acrylate, 2-aminoethyl methacrylate, 2-(N,N-dimethylamino)ethyl acrylate, 2-(N,N-dimethylamino)ethyl methacrylate, 2-(N,N-diethylamino)ethyl methacrylate, or 2-diisopropylaminoethyl methacrylate.

In some examples, the polyionic chains have a structure represented by Formula I, II or III:

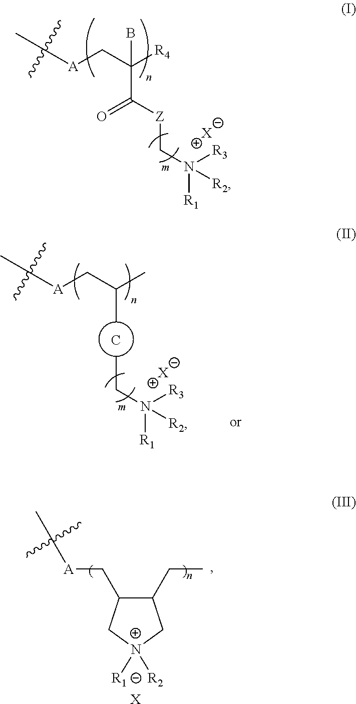

wherein
the squiggly line depicts the point of attachment to surface of the coated polymer particle core;
A is an anchor group selected from —O—, —S—, —C(O)—, —OC(O)—, optionally substituted C₁-C₄ alkyl, C₁-C₄ thioalkyl, amino, C₁-C₄ alkylamine, C₁-C₄ alkylamide, or C₁-C₄ alkyl ester group;
B is H, halogen, or optionally substituted C₁-C₄ alkyl or carbonotrithioate group;
C is aryl or heteroaryl;
n is degree of polymerization ranging from 10-150 repeating monomer units;
Z is NH or O, or Z and one of R₁, R₂, and R₃, together with the atoms to which they are attached, form a C₄-C₆ heterocyclic;
m is from 1-10;
R₁, R₂, and R₃ are each independently a lone pair, H, or optionally substituted C₁-C₄ alkyl, or R₁, and R₂, together with the atoms to which they are attached, form a C₃-C₆ heterocyclic; R₄ is H, halogen or methyl; and
X is a counter ion, provided that when one of R₁, R₂, and R₃ in Formula I or II is a lone pair, or
R₁ or R₂ in Formula III is a lone pair, X is absent.

In some examples, A is an optionally substituted C₁-C₄ alkyl or C₁-C₄ alkyl ester group. In some examples, A is an optionally substituted C₁-C₄ thioalkyl group.

In some examples, A is

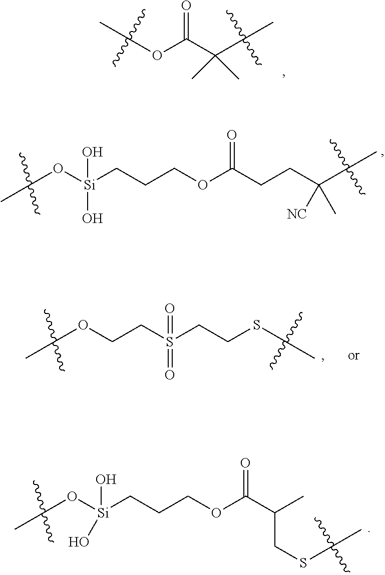

In some examples, including all the previous examples B is hydrogen or methyl. In some examples, B is methyl.

In some examples, including all the previous examples, C is phenyl or C₅-C₆ heteroaryl (e.g., imidazolyl and pyridyl). In some further examples, C is C₅-C₆ heteroaryl.

In some examples, including all the previous examples, the polyionic chains are of Formula I or II, and R₁, R₂, and R₃ are each independently H, methyl, ethyl, isopropyl, t-butyl, or benzyl. In some examples, the polyionic chains are of Formula I or II, and one of R₁, R₂, and R₃ is a lone pair.

In some examples, the polyionic chains are of Formula III, and R₁ and R₂ are each independently H, methyl, ethyl, isopropyl, t-butyl, or benzyl. In some examples, the polyionic chains are of Formula III, and R₁ or R₂ is a lone pair.

In some examples, including all the previous examples, the polyionic chains have any one of structures:

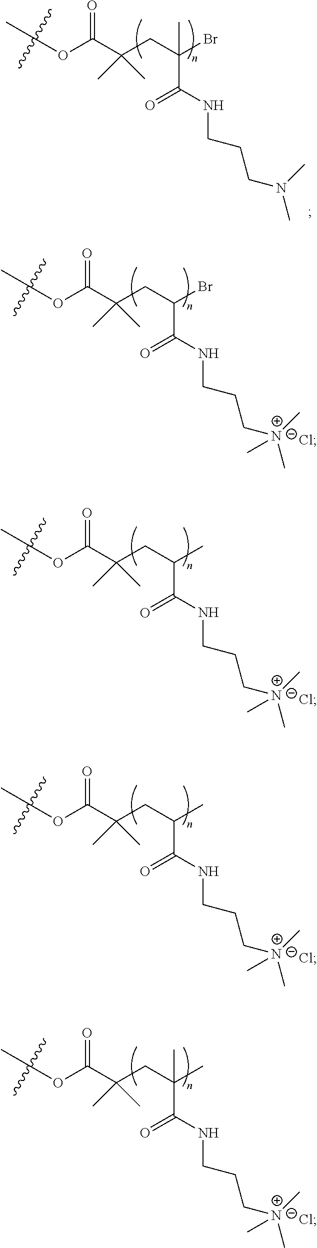

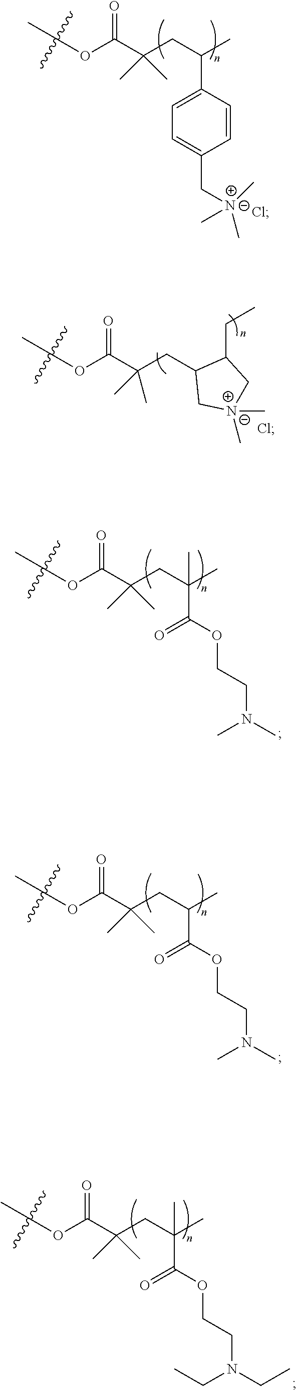

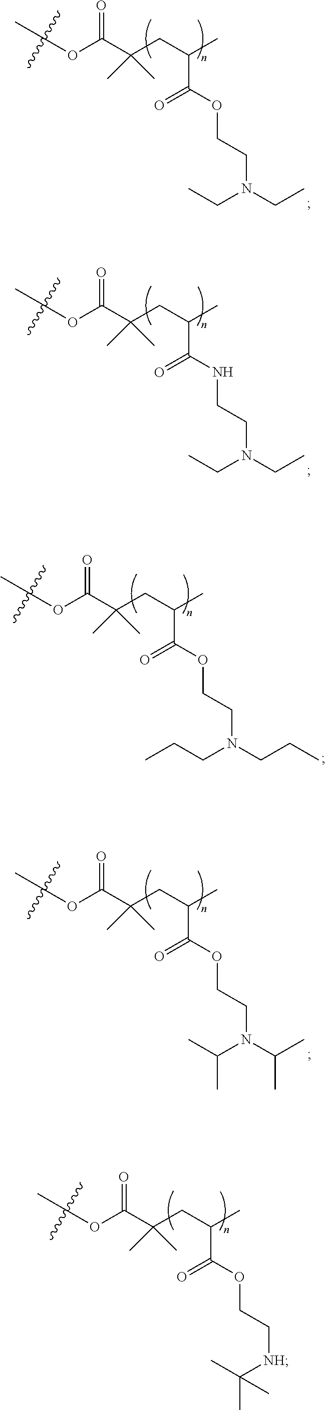

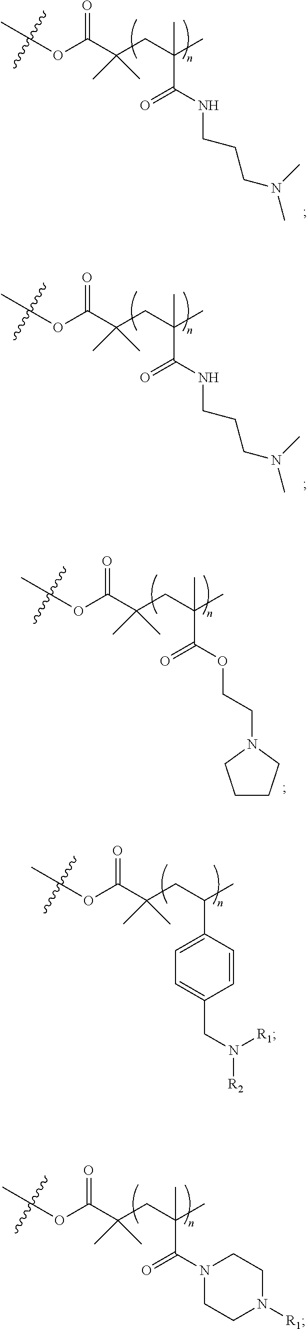

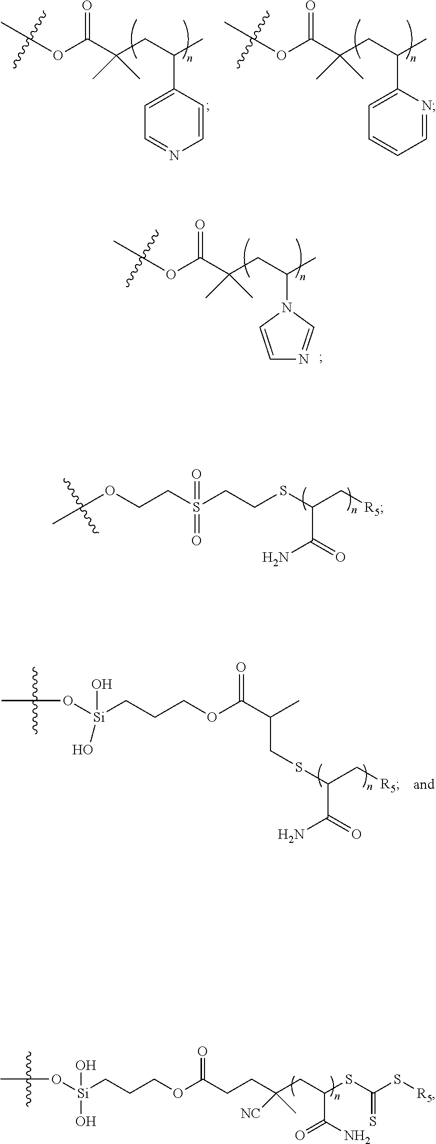

wherein R₅ is optionally substituted C₁-C₄ alkyl.

In some examples, the polyionic chains are strong anion exchangers (FIG. 1D), weak anion exchangers (FIG. 1E), or a mixture thereof.

The surface-grafted polyionic chains generated via various living polymerization techniques that allow control and variation of polymer compositions and structures, as illustrated FIG. 1B.

Exemplary control and characterization of structural properties of the surface-grafted polyionic chains according to the present disclosure are provided in Table 1, below.

TABLE 1
Control and characterization of structural properties of grafted polymers.

| Composition | Chain length | Grafting density |
|---|---|---|
| [Monomer₁]:[Monomer₂] | [Initiator]₀:[Monomer]₀ | [Particle]₀:[Initiator]₀ |
| 1:0 to 5:1 | 10 to 250 repeat units | 0.7 to 15 μmol chains/m² |
| Ionic capacity measurement, % N | Ionic capacity measurement, % N | $σ = \frac{{m}_{polymer} {N}_{A}}{{M}_{n} \cdot SA}$ |

Living polymerization techniques that may be used to prepare the surface-grafted polyionic chains of the present disclosure are known in the art. Exemplary living polymerization techniques include reversible addition fragmentation chain transfer (RAFT), atom transfer radical polymerization (ATRP), ring-opening polymerization (ROP), ring-opening metathesis polymerization (ROMP), nitroxide mediated polymerization (NMP), supplemental activator and reducing agent (SARA)-ATRP), activators regenerated by electron transfer (ARGET)-ATRP, etc. See, for example, Grubbs and Grubbs, Macromolecules 2017, 50 (18): 6979-6997.

In some examples, the polyionic chains of the present disclosure form homopolymers (e.g., chains of a single type of polymer), di-block copolymers, di-block mixed mode ion exchangers, random mixed ion exchangers, gradient ion exchangers, multi-component ion exchangers, or bottlebrush ion exchangers. See, e.g., FIG. 1B. Using living polymerization techniques, the surface-grafted polymeric chains can be tailored to a desired structure, morphology, density, and/or functional make-up.

In some examples, compositions of the present disclosure have grafting densities between 0.5-15 μmol chains/m². In some examples, compositions of the present disclosure comprise chain lengths between 10 to 250 repeat units.

### Chromatographic Devices

In some aspects of the present disclosure, the non-porous particle cores described herein may be provided in a suitable chromatographic device. For this purpose, the non-porous particle cores described herein may be provided in conjunction with a suitable housing. The non-porous particle cores and the housing may be supplied independently, or the non-porous particle cores may be pre-packaged in the housing. Housings for use in accordance with the present disclosure commonly include a chamber for accepting and holding the non-porous particle cores. In various examples, the housings may be provided with an inlet and an outlet leading to and from the chamber.

Suitable construction materials for the chromatographic housings include inorganic materials, for instance, metals such as stainless steel and ceramics such as glass, as well as synthetic polymeric materials such as polyethylene, polypropylene, polyether ether ketone (PEEK), and polytetrafluoroethylene, among others.

In certain examples, the chromatographic housings may include one or more filters which act to hold the non-porous particle cores in a housing. Exemplary filters may be, for example, in a form of a membrane, screen, frit or spherical porous filter.

In certain examples, the chromatographic device is a chromatographic column.

The present disclosure also provides for a kit comprising the non-porous particle cores, housings or devices as described herein and instructions for use. In one embodiment, the instructions are for use with a separation device, e.g., a chromatographic column.

### Chromatographic Separations

In some aspects of the present disclosure, the non-porous particle cores can be used in a variety of chromatographic separation methods. As such, the chromatographic devices and chromatographic kits described herein can also be utilized for such methods. Examples of chromatographic separation methods in which the non-porous particle cores of the present disclosure can be used include both high-pressure liquid chromatography (HPLC) and ultra-high pressure liquid chromatography (UHPLC).

The non-porous particle cores, devices and kits of the present disclosure may be used for chromatographic separations of small molecules, carbohydrates, antibodies, whole proteins, peptides, and/or DNA, among other species.

Such chromatographic separations may comprise loading a sample onto the non-porous particle cores in accordance with the present disclosure and eluting adsorbed species from the non-porous particle cores with a mobile phase.

Such chromatographic separations may be performed in conjunction with a variety of aqueous and/or organic mobile phases (i.e., in mobile phases that contain water, an organic solvent, or a combination of water and organic solvent) and in conjunction with a variety of mobile phase gradients, including solvent species gradients, temperature gradients, pH gradients, salt concentration gradients, or gradients of other parameters.

In some examples, the chromatographic method comprises: (a) loading a sample onto a chromatographic column comprising the composition of the present disclosure and (b) flowing a mobile phase through the column.

In some examples, wherein the sample comprises large nucleic acids (e.g., deoxyribonucleic acid (DNA), and ribonucleic acid (RNA)). In some examples, the sample comprises single stranded RNA. In some examples, the sample comprises DNA fragments.

### EXAMPLES

### General Methods

The particle core, composed of non-porous crosslinked polystyrene-divinylbenzene, a p(GMA-EDMA) primer layer, and a hydrophilic coating for further surface functionalization, was prepared according to know methods, for example, as described in WO2019168989A1, the disclosure of each of which is incorporated herein by reference.

A two-step process was used for the synthesis of polymeric anion exchange particles. The first step involved the covalent attachment of initiator groups onto the hydrophilic layer of the particles. These groups serve as initiation sites where polymers can grow when using a grafting-from technique, or as reactive sites where pre-synthesized polymers can be coupled when using a grafting to approach. Several living/controlled radical polymerization techniques such as atom transfer radical polymerization (ATRP), reversible addition-fragmentation chain transfer (RAFT) polymerization, ring-opening polymerization (ROP), nitroxide-mediated polymerization (NMP), and ring-opening metathesis polymerization (ROMP) can be utilized to achieve well-defined, narrowly dispersed grafted polymers on surfaces (Zoppe, J., et al., Chem. Rev. 117:1105-1318 (2017). The second step involves controlled grafting of polymeric chains bearing anion exchange functionalities to and/or from the initiator-modified hydrophilic layer of particles.

### Materials

Reagents were purchased from commercial sources and used directly without purification unless noted otherwise. 1:1 (v/v) dimethylformamide (DMF)/water solvent was deoxygenated by sparging with nitrogen for at least 45 minutes. Monomers such as (3-acrylamide propyl)trimethyl ammonium chloride (APTAC), N-[3-(N,N-dimethylamino) propyl]acrylamide (DMAPA), (vinylbenzyl)trimethylammonium chloride (VBTMAC), 2-(dimethylamino)ethyl methacrylate, 2-(dimethylamino)ethyl acrylate, 2-(diethylamino)ethyl methacrylate were passed through a column packed with activated carbon prior to polymerization to remove inhibitors. CuBr was purified by stirring with glacial acetic acid for 24 hours (h) at room temperature, followed by rinsing with ethanol and then drying overnight in vacuum. MILLIQ® water (18 m (2 cm resistivity) was used for all syntheses.

### Characterization

The % C, % H, % N values were measured by combustion analysis (CE-440 Elemental Analyzer; Exeter Analytical Inc., North Chelmsford, MA). The specific surface areas (SSA), specific pore volumes (SPV) and the average pore diameters (APD) of these materials were measured using the multi-point N2 sorption method (Micromeritics ASAP 2400; Micromeritics Instruments Inc., Norcross, GA). The SSA was calculated using the BET method, the SPV was the single point value determined for P/P0>0.98 and the APD was calculated from the desorption leg of the isotherm using the BJH method. Scanning electron microscopic (SEM) image analyses were performed (JEOL JSM-5600 instrument, Tokyo, Japan) at 7 kV. Particle sizes were measured using a Beckman Coulter Multisizer 3 analyzer (30 μm aperture, 70,000 counts; Miami, FL). The particle diameter (dp) was measured by SEM or by using a Beckman Coulter as the 50% cumulative diameter of the volume-based particle size distribution. The width of the distribution was measured as the 90% cumulative volume diameter divided by the 10% cumulative volume diameter (denoted dv90/dv10 ratio). Ionic capacity in the ion-exchangers was determined by acid-base titration.

### Example 1. Surface Functionalization of Hydrophilic Coated Particles with ATRP Initiators (SI-ATRP)

100 g hydrophilic coated particles were washed with anhydrous DMF solution to replace traces of water in its surface with DMF completely. Then, in a clean, dry 1-L beaker, 100 g drained hydrophilic coated particles were dispersed in 400 mL anhydrous DMF using horn sonication for 2 minutes with pulse mode: 2 seconds on, 1 second off. The slurry was then transferred to a 1-L three-necked round bottom flask equipped with condenser, thermometer, heating mantle, mechanical stirring and N₂ purge, using 267 mL anhydrous DMF to quantitatively transfer the contents of the beaker to the flask. Triethylamine (39.7 mL) was added to the flask. The reaction mixture was allowed to stir at 0° C. using an ice water bath. Once the temperature reached 0° C., 6.70 mL α-bromoisobutyryl bromide was added to the flask and the reaction mixture was allowed to stir at 0° C. for 30 minutes before raising the temperature to 30° C. The reaction was allowed to proceed for 16 h at 30° C. After the reaction, the material particles were collected via filtration and washed sequentially with excess DMF, ethanol, and distilled water, and dried over nitrogen for at least 20 minutes. The surface-functionalized particles were then stored at 2-8° C. prior to use in the next steps. (% Br=0.30)

### Example 2. Synthesis of Strong Anion Exchanger (SAX) a (pAPTAC) Via SI-ATRP

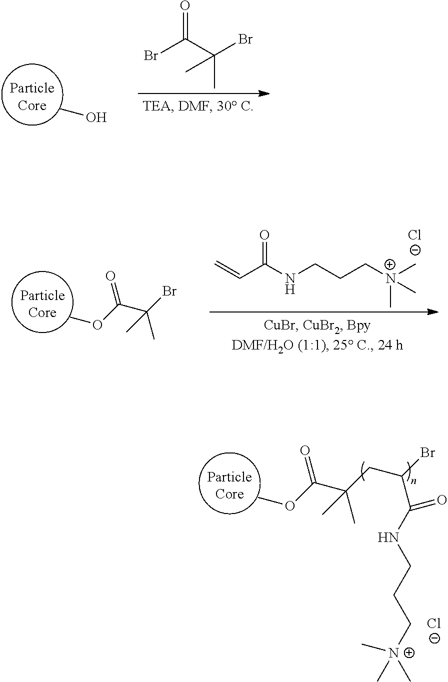

In a clean, dry 100-mL beaker, 16 g particles were dispersed in 50 mL deoxygenated 1:1 (v/v) DMF/water using horn sonication for 2 minutes with pulse mode: 2 seconds on, 1 second off. The slurry was then transferred to a 150-mL three-necked round bottom flask equipped with condenser, thermometer, heating mantle, mechanical stirring, and N₂ purge, using additional 70 mL 1:1 (v/v) DMF/water to quantitatively transfer the contents of the beaker. Then, 44.6 g APTAC was added to the reaction flask, and the contents were deoxygenated by sparging with nitrogen for at least 45 minutes. In a clean vial, 0.0268 g Copper (II) bromide (CuBr₂) and 0.7497 g bipyridyl were dissolved in 5 mL deoxygenated 1:1 (v/v) DMF/water. The resulting CuBr₂-bipyridyl solution was then added to the reaction flask. The polymerization was initiated by adding 0.3456 g CuBr to the reaction flask, and the reaction was allowed to proceed for 20 h at room temperature under nitrogen. Upon completion of the reaction, the particles were isolated by filtration and subsequently washed with 0.1M ethylenediaminetetraacetic acid (EDTA), water, 1M HNO₃, water, 1M HCl, water and methanol. The particles were dried in a vacuum oven at 45° C. for 20 h. (% N: 0.96, IC: 157 μmol/g).

SAX A2 and SAX A3 were synthesized according to the above procedure. The characterization data for SAX A1, SAX A2, and SAX A3 are provided in Table 2, below.

TABLE 2
Examples of strong anion exchangers synthesized using SI-ATRP.

| | Synthesis and characterization | | | | | | | | |
|---|---|---|---|---|---|---|---|---|---|
| | | | | Ionic | 1.5 mg/mL glycine in 20 mM sodium | | | | |
| | | | | capacity | phosphate buffer pH 6.8 | | | | |
| | Initiator | | | from | | | | | Column |
| | content | | % | titration | | USP | USP | | press. |
| Example | (% Br) | [I]₀:[M]₀ | N | (μmol/g) | RT | Plates | Tailing | RPH | (psi) |
| SAX | 0.30 | 1:50 | 0.77 | 91 | 1.493 | 5493 | 1.267 | 3.034 | 432 |
| A1 | | | | | | | | | |
| SAX | 0.30 | 1:150 | 0.85 | 137 | 1.487 | 6285 | 1.227 | 2.652 | 586 |
| A2 | | | | | | | | | |
| SAX | 0.30 | 1:200 | 0.96 | 157 | 1.609 | 5296 | 1.126 | 3.147 | 1182 |
| A3 | | | | | | | | | |

Table 2 summarizes the strong anion exchangers and the convenient control of ionic capacity of the resulting exchanger with varying the monomer to initiator ratio during the synthesis process. Additionally, columns packed with SAX particles showed good efficiency with glycine and acceptable column backpressure.

### Example 3. Synthesis of SAX B (pVTMAC) Via SI-ATRP

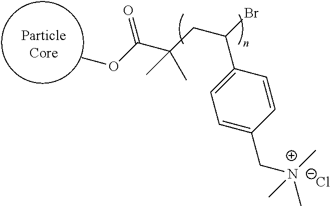

In a clean, dry 100-mL beaker, 16 g particles were dispersed in 50 mL deoxygenated 1:1 (v/v) DMF/water using horn sonication for 2 minutes with pulse mode: 2 seconds on, 1 second off. The slurry was then transferred to a 150-mL three-necked round bottom flask equipped with condenser, thermometer, heating mantle, mechanical stirring and N₂ purge, using additional 70 mL 1:1 (v/v) DMF/water to quantitatively transfer the contents of the beaker. 34.3003 g VBTMAC was added to the reaction flask and the contents were deoxygenated by sparging with nitrogen for at least 45 minutes. In a clean vial, 0.0268 g CuBr₂ and 0.7497 g bipyridyl were dissolved in 5 mL deoxygenated 1:1 (v/v) DMF/water. CuBr₂-bipyridyl solution was then added to the reaction flask. The polymerization was initiated by adding 0.3443 g CuBr to the reaction flask, and the reaction was allowed to proceed for 20 h at room temperature under nitrogen. Upon completion of the reaction, the particles were isolated by filtration and washed with 0.1M EDTA, water, 1M HNO₃, water, 1M HCl, water and methanol. The particles were dried in a vacuum oven at 45° C. for 20 h. (% N: 0.70, IC: 169 μmol/g).

SAX B2 was synthesized according to the above procedure. Characterization data for SAX B1 and SAX B2 are provided in Table 3, below.

TABLE 3
Additional examples of strong anion exchangers synthesized using SI-ATRP.

| | Synthesis and characterization | | | | | | | | |
|---|---|---|---|---|---|---|---|---|---|
| | | | | Ionic | 1.5 mg/mL glycine in 20 mM sodium | | | | |
| | | | | capacity | phosphate buffer pH 6.8 | | | | |
| | Initiator | | | from | | | | | Column |
| | content | | % | titration | | USP | USP | | press. |
| Example | (% Br) | [I]₀:[M]₀ | N | (μmol/g) | RT | Plates | Tailing | RPH | (psi) |
| SAX | 0.30 | 1:100 | 0.67 | 157 | 1.548 | 7442 | 1.261 | 2.239 | 398 |
| B1 | | | | | | | | | |
| SAX | 0.30 | 1:100 | 0.70 | 179 | 1.588 | 7077 | 1.302 | 2.355 | 505 |
| B2 | | | | | | | | | |

Table 3 summarizes the strong anion exchangers and the convenient control of ionic capacity of the resulting exchanger with varying the monomer to initiator ratio during the synthesis process. Additionally, columns packed with SAX particles showed good efficiency with glycine and acceptable column backpressure.

### Example 4. Synthesis of Weak Anion Exchanger (WAX) a (pDMAPA) Via SI-ATRP

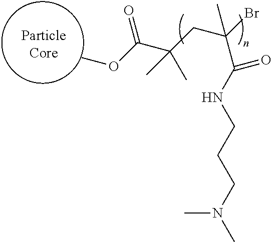

In a clean, dry 100-mL beaker, 16 g particles were dispersed in 50 mL deoxygenated 1:1 (v/v) DMF/water using horn sonication for 2 minutes with pulse mode: 2 seconds on, 1 second off. The slurry was then transferred to a 150-mL three-necked round bottom flask equipped with condenser, thermometer, heating mantle, mechanical stirring and N₂ purge, using additional 70 mL 1:1 (v/v) DMF/water to quantitatively transfer the contents of the beaker. 7.1696 g DMAPA was added to the reaction flask and the contents were deoxygenated by sparging with nitrogen for at least 45 minutes. In a clean vial, 0.0268 g CuBr₂ and 0.7497 g bipyridyl were dissolved in 5 mL deoxygenated 1:1 (v/v) DMF/water. CuBr₂-bipyridyl solution was then added to the reaction flask. The polymerization was initiated by adding 0.3443 g CuBr to the reaction flask, and the reaction was allowed to proceed for 20 h at room temperature under nitrogen. Upon completion of the reaction, the particles were isolated by filtration and washed with 0.1M EDTA, water, 1M HNO₃, water, 1M HCl, water and methanol. The particles were dried in a vacuum oven at 45° C. for 20 h. (% N: 1.02, IC: 140 μmol/g).

WAX A2 and WAX A3 were synthesized according to the above procedure. The characterization data for WAX A1, WAX A2, and WAX A3 are provided in Table 4, below.

TABLE 4
Examples of weak anion exchangers synthesized using SI-ATRP.

| | Synthesis and characterization | | | | | | | | |
|---|---|---|---|---|---|---|---|---|---|
| | | | | Ionic | 1.5 mg/mL glycine in 20 mM sodium | | | | |
| | | | | capacity | phosphate buffer pH 6.8 | | | | |
| | Initiator | | | from | | | | | Column |
| | content | | % | titration | | USP | USP | | press. |
| Example | (% Br) | [I]₀:[M]₀ | N | (μmol/g) | RT | Plates | Tailing | RPH | (psi) |
| WAX | 0.30 | 1:75 | 0.81 | 129 | 1.605 | 6449 | 1.343 | 2.584 | 225 |
| A1 | | | | | | | | | |
| WAX | 0.30 | 1:150 | 1.02 | 140 | 1.411 | 6641 | 1.276 | 2.510 | 904 |
| A2 | | | | | | | | | |
| WAX | 0.30 | 1:250 | 1.23 | 232 | 1.417 | 4467 | 1.171 | 3.731 | 1411 |
| A3 | | | | | | | | | |

Table 4 summarizes the weak anion exchangers and the convenient control of ionic capacity of the resulting exchanger with varying the monomer to initiator ratio during the synthesis process. Additionally, columns packed with WAX particles showed good efficiency with glycine and acceptable column backpressure.

### Example 5. Synthesis of WAX B (pDEAEMA) Via SI-ATRP

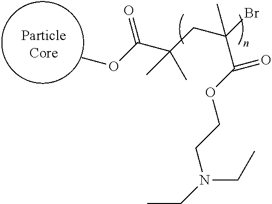

In a clean, dry 100-mL beaker, 16 g particles were dispersed in 60 mL deoxygenated 1:1 (v/v) water/ethanol using horn sonication for 2 minutes with pulse mode: 2 seconds on, 1 second off. In a 150-mL three-necked round bottom flask equipped with condenser, thermometer, heating mantle, mechanical stirring and N₂ purge, 5.5578 g 2-(diethylamino)ethyl methacrylate (DEAEMA), 1.0567 g ascorbic acid, and 0.1340 g CuBr₂ were dissolved in 1:1 (v/v) water/ethanol and the contents were deoxygenated by sparging with nitrogen for at least 45 minutes. The polymerization was initiated by adding the dispersed particles to the reaction flask, and the reaction was allowed to proceed for 20 h at room temperature under nitrogen. Upon completion of the reaction, the particles were isolated by filtration and washed with 0.1M EDTA, water, 1M HNO₃, water, 1M HCl, water and methanol. The particles were dried in a vacuum oven at 45° C. for 20 h. (% N: 0.78, IC: 117 μmol/g).

### Example 6. Synthesis of Random Mixed-Mode Anion Exchanger (p (APTAC-r-AM)) Via SI-ATRP

In a clean, dry 100-mL beaker, 16 g particles were dispersed in 50 mL deoxygenated 1:1 (v/v) DMF/water using horn sonication for 2 minutes with pulse mode: 2 seconds on, 1 second off. The slurry was then transferred to a 150-mL three-necked round bottom flask equipped with condenser, thermometer, heating mantle, mechanical stirring and N₂ purge, using additional 70 mL 1:1 (v/v) DMF/water to quantitatively transfer the contents of the beaker. A solution containing 11.16 g APTAC, and 2.88 g acrylamide was added to the reaction flask and the contents were deoxygenated by sparging with nitrogen for at least 45 minutes. In a clean vial, 0.0268 g CuBr₂ and 0.7497 g bipyridyl were dissolved in 5 mL deoxygenated 1:1 (v/v) DMF/water. CuBr₂-bipyridyl solution was then added to the reaction flask. The polymerization was initiated by adding 0.3456 g CuBr to the reaction flask, and the reaction was allowed to proceed for 20 h at room temperature under nitrogen. Upon completion of the reaction, the particles were isolated by filtration and washed with 0.1M EDTA, water, 1M HNO₃, water, 1M HCl, water and methanol. The particles were dried in a vacuum oven at 45° C. for 20 h. (% N: 0.96, IC: 157 μmol/g).

### Example 7. Diblock Mixed Mode Anion Exchanger p (APTAC-b-DMAPA) Via SI-ATRP

In a clean, dry 100-mL beaker, 16 g p (APTAC)-grafted particles were dispersed in 50 mL deoxygenated 1:1 (v/v) DMF/water using horn sonication for 2 minutes with pulse mode: 2 seconds on, 1 second off. The slurry was then transferred to a 150-mL three-necked round bottom flask equipped with condenser, thermometer, heating mantle, mechanical stirring and N₂ purge, using additional 70 mL 1:1 (v/v) DMF/water to quantitatively transfer the contents of the beaker. Then, 7.1696 g DMAPA was added to the reaction flask and the contents were deoxygenated by sparging with nitrogen for at least 45 minutes. In a clean vial, 0.0268 g CuBr₂ and 0.7497 g bipyridyl were dissolved in 5 mL deoxygenated 1:1 (v/v) DMF/water. CuBr₂-bipyridyl solution was then added to the reaction flask. The polymerization was initiated by adding 0.3443 g CuBr to the reaction flask, and the reaction allowed to proceed for 20 h at room temperature under nitrogen. Upon completion of the reaction, the particles were isolated by filtration and washed with 0.1M EDTA, water, 1M HNO₃, water, 1M HCl, water and methanol. The particles were dried in a vacuum oven at 45° C. for 20 h.

### Example 8. Multi-Component Anion Exchanger Via RAFT and Azide-Alkyne Click Reaction

RAFT Polymerization of pDMAPA/pAPTAC

In a clean, dry beaker, 30.0 g monomer was dissolved in 162 mL 2:1 (v/v) acidic water: 2-propanol solution and transferred in a 250-mL three-necked round bottom flask equipped with condenser, thermometer, heating mantle, mechanical stirring and N₂ purge. Then, 0.90 g chain transfer agent (CTA) and 0.45 g radical initiator were added to the flask. The contents of the flask were deoxygenated by sparging pure nitrogen for 45 minutes. The reaction flask was then sealed, and the reaction was initiated by heating the mixture to 70° C. The polymerization was allowed to proceed for 24 h. The reaction was quenched by allowing the solution to cool to room temperature. The polymer was purified by precipitation from acetone (six times). The recovered polymer was dried under vacuum at 45° C. overnight.

### Azide-Modified Hydrophilic Coated Particles

In a clean, dry beaker, 15 g Br-modified hydrophilic coated particles were dispersed in 100 mL anhydrous DMF using horn sonication for 2 minutes with pulse mode: 2 seconds on, 1 second off. The slurry was then transferred to a 250-mL three-necked round bottom flask equipped with condenser, thermometer, heating mantle, mechanical stirring and N₂ purge, using additional 50 mL anhydrous DMF to quantitatively transfer the contents of the beaker. Then, 1.17 g sodium azide was added. The reaction was allowed to proceed at 80° C. for 18 h.

### Alkyne Modification of RAFT Homopolymers

In a clean, dry beaker, 15.0 g pAPTAC was dissolved in 40 mL anhydrous DMF and transferred in a 100-mL three-necked round bottom flask equipped with condenser, thermometer, heating mantle, mechanical stirring and N₂ purge, using additional 35 mL DMF to quantitatively transfer all contents of the beaker to the flask. Then, 276.8 mg N-(3-dimethylaminopropyl)-N′-ethylcarbodiimide hydrochloride (EDC HCl) and 97.6 g propargyl alcohol were added to the reaction flask. A solution containing 17.7 mg 4-dimethylaminopyridine (DMAP) and 5 mL DMF was slowly added to the reaction mixture. The reaction was allowed to proceed for 48 h at room temperature. After the reaction, the polymer was recovered by precipitation from ethanol and acetone. The polymers were dried under vacuum overnight at room temperature.

### Synthesis of Multi-Component Ion Exchanger Via Azide-Alkyne Click Reaction

In a clean, dry beaker, 10.0 g azide-modified particles and alkyne-modified homopolymer were dispersed in 50 mL 1:1 (v/v) water:DMF by horn sonication (pulse 2 seconds on, 1 second off). The slurry was transferred to a 150-mL three-necked round bottom flask equipped with condenser, thermometer, heating mantle, mechanical stirring and N₂ purge, using additional 50 mL solvent to quantitatively transfer the contents of the beaker to the flask. The reaction mixture was deoxygenated by sparging with nitrogen for at least one hour. A catalyst stock solution containing CuBr and N,N,N′,N″,N″-pentamethyldiethylenetriamine (PMDETA) was prepared and deoxygenated by sparging with nitrogen for at least 20 minutes. The reaction was initiated by adding catalyst stock solution to the reaction flask and allowing the contents of the flask to stir at 40° C. for 18 h under nitrogen. Upon completion of the reaction, the particles were filtered and washed subsequently with DMF, water, EDTA, nitric acid solution, water, and methanol. The particles were dried under vacuum overnight at 45° C.

### Example 9. Bottlebrush Anion Exchanger p (BIEM-g-APTAC) Via RAFT-ARTP

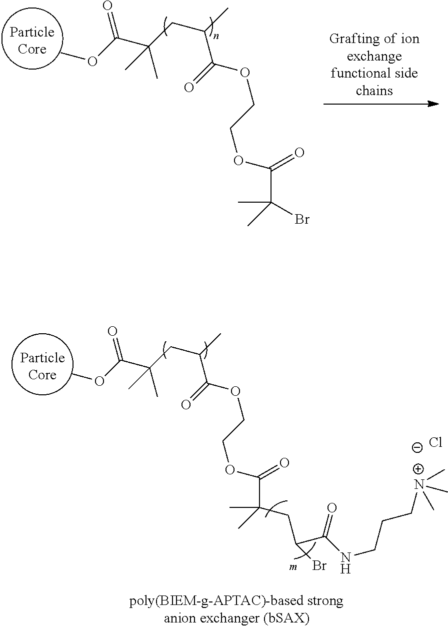

### RAFT-Functionalized Hydrophilic Coated Particles

In a clean, dry beaker, 25.0 g hydrophilic coated particles were dispersed in 50 mL anhydrous DMF and transferred to a 150-mL three-necked round bottom flask equipped with condenser, heating mantle, mechanical stirring, and N₂ purge, using additional 50 mL anhydrous DMF to quantitatively transfer the contents of the beaker to the flask. 149.5 mg DMAP and 75.7 mg DCC were added to the flask and the mixture was allowed to stir for 1 h at 0° C. After an hour, the reaction mixture was raised to room temperature and continued to stir for 20 h. After the reaction, the particles were filtered and washed with ethanol, THF and water. The particles were dried under vacuum at room temperature overnight.

### RAFT Polymerization of (2-(Bromoisobutyryl)Ethyl Methacrylate (BIEM)

RAFT-functionalized particles were dispersed in anhydrous DMF and transferred to a 150-mL three-necked round bottom flask equipped with condenser, heating mantle, mechanical stirring, and N₂ purge. Then, BIEM was added to the reaction flask. Subsequently, a mixture of AIBN in DMF was added, and the flask was then degassed by sparing with nitrogen for 40 minutes before stirring the mixture at 65° C. After 30 hours, the flask was cooled in a water bath to room temperature, and the particles were filtered and subsequently washed with DMF, ethanol, water, and methanol. The particles were dried under vacuum overnight at 45° C.

Synthesis of Brush pAPTAC Via ATRP

In a clean, dry 100-mL beaker, 16 g pBIEM-grafted particles were dispersed in 50 mL deoxygenated 1:1 (v/v) DMF/water using horn sonication for 2 minutes with pulse mode: 2 seconds on, 1 second off. The slurry was then transferred to a 150-mL three-necked round bottom flask equipped with condenser, thermometer, heating mantle, mechanical stirring and N₂ purge, using additional 70 mL 1:1 (v/v) DMF/water to quantitatively transfer the contents of the beaker. Then, 44.6 g APTAC was added to the reaction flask and the contents were deoxygenated by sparging with nitrogen for at least 45 minutes. In a clean vial, 0.0268 g CuBr₂ and 0.7497 g bipyridyl were dissolved in 5 mL deoxygenated 1:1 (v/v) DMF/water. CuBr₂-bipyridyl solution was then added to the reaction flask. The polymerization was initiated by adding 0.3456 g CuBr to the reaction flask, and the reaction was allowed to proceed for 20 h at room temperature under nitrogen. Upon completion of the reaction, the particles were isolated by filtration and subsequently washed with 0.1M EDTA, water, 1M HNO₃, water, 1M HCl, water and methanol. The particles were dried in a vacuum oven at 45° C. for 20 h.

### Example 10: DNA Ladder Separation: Protein-Pak™ Hi Res Q Vs SAX A3

The chromatographic performance of SAX A3 was investigated by ion exchange separation of double-stranded (ds) DNA ladder (10 kilobase pairs (kbp)) using an ACQUITY UPLC H-Class Bio System, using NaCl as the eluting salt in AEX separation, and compared to that of Protein-a Pak™ Hi Res Q under identical conditions.

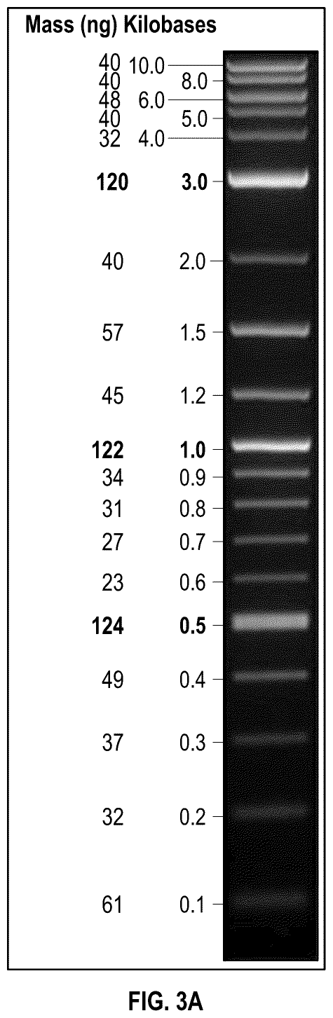

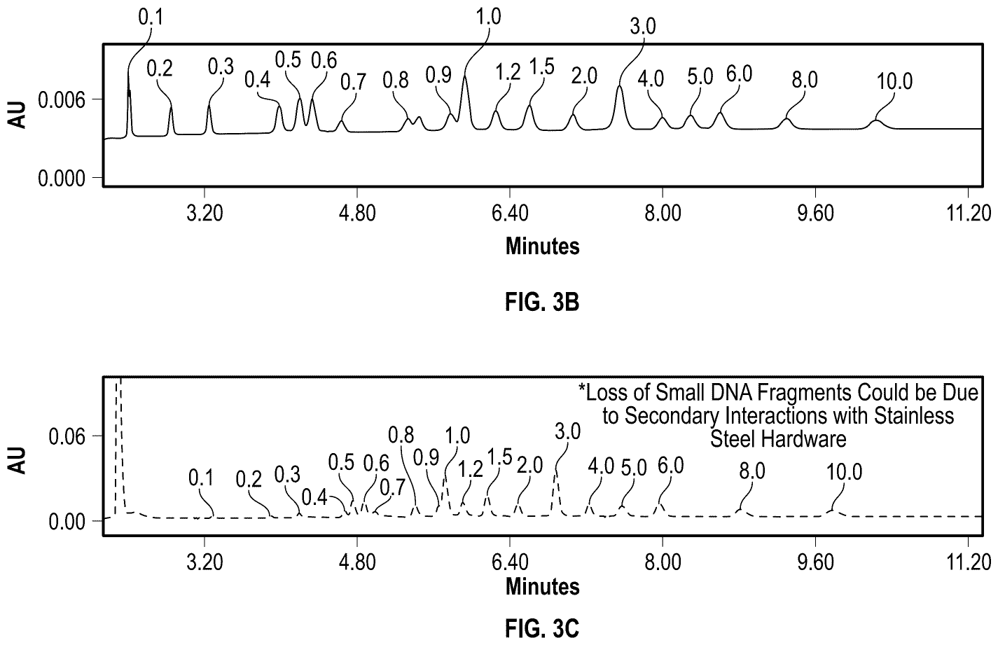

As illustrated in FIG. 3A-FIG. 3C, the evaluation of the SAX A3 column provided respectable results for size-based separation of DNA fragments. The chromatogram obtained with the SAX A3 column (FIG. 3C) was comparable to the one obtained with the PROTEIN-PAK™ Hi Res Q column (FIG. 3B) (5 μm, 4.6×100 mm). Loss of small DNA fragments with the SAX A3 column were likely due to secondary interactions with stainless steel hardware.

### Example 11. Evaluations of SAX and WAX Columns Using dsDNA Ladder Separation

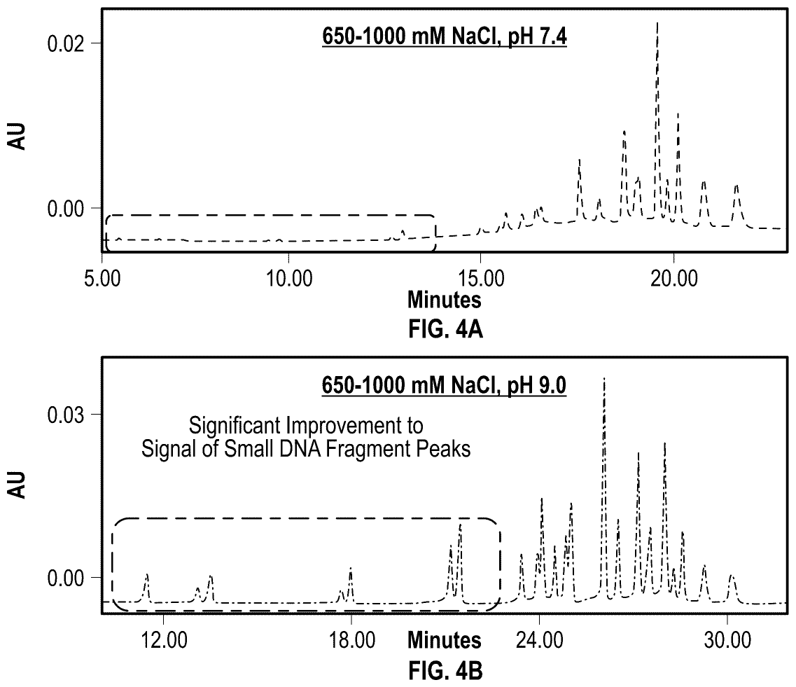

The chromatographic performance of a series of strong and weak anion exchangers was investigated by ion exchange separation of dsDNA ladder (10 kbp) using an ACQUITY UPLC H-Class Bio System, using NaCl as the eluting salt in AEX separation. The use of NaCl as eluting salt in place of tetramethylammonium chloride (TMAC) resulted in comparable chromatography between both pAPTAC (SAX) and pDMAPA (WAX) materials, with the benefit of more congruent gradient concentrations across materials. Increasing Tris buffer pH from 7.4 to 9 increased sensitivity for small DNA fragments on APTAC prototype and significantly lowered the starting salt concentration necessary for DMAPA material, allowing for a shorter test method (FIG. 4A-FIG. 4B and FIG. 51-FIG. 5B). A unified test method was developed and optimized on high ionic capacity APTAC and DMAPA columns. High ionic capacity ATRP grafted APTAC & DMAPA prototypes offered the best resolution for size-based separation of DNA fragments.

As demonstrated in FIG. 4A-FIG. 4B, a significant improvement in signal of small DNA fragment peaks was observed in the SAX (IC: 157 μmol/g) chromatogram with the more basic eluent (650-1000 mM NaCl, pH 9.0).

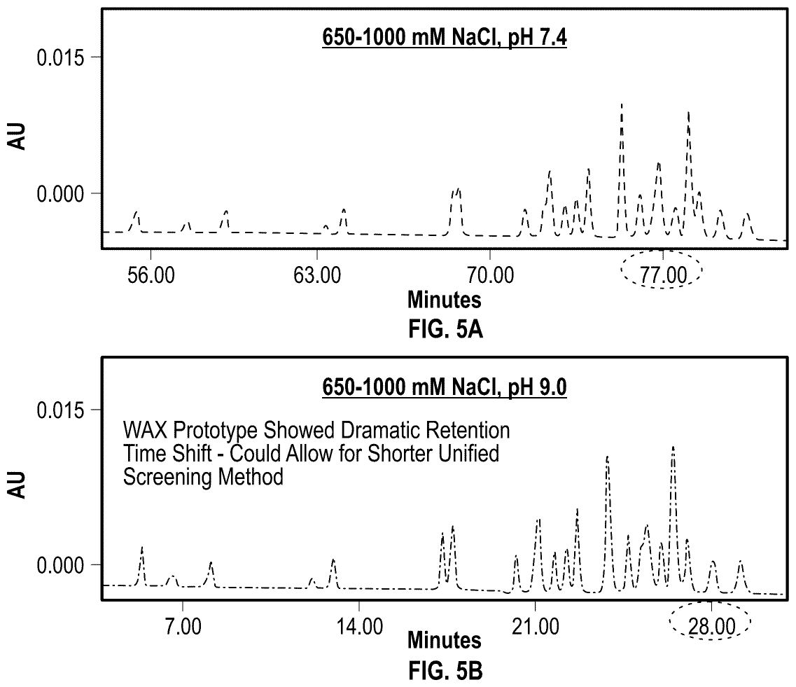

As demonstrated in FIG. 5A-FIG. 5B, a dramatic retention time shift was observed in the WAX (IC: 140 μmol/g) chromatogram with the more basic eluent (650-1000 mM NaCl, pH 9.0). This observed retention time shift could allow for shorter unified screening methods.

### Example 12. Grafting-to Synthesis of HILIC Silica Using RAFT and Thiol-Ene Click Reaction

### RAFT Polymerization of Acrylamide

In a clean, dry beaker, 10.0 g acrylamide monomer were dissolved in 70.0 mL dimethyl sulfoxide (DMSO), and the resulting mixture was transferred to a 100-mL three-necked round bottom flask equipped with condenser, thermometer, heating mantle, mechanical stirring and

Argon purge. Then, 49.1 mg CTA (chain transfer agent) and 12.3 mg 4,4′-azobis(4-cyanovaleric acid) radical initiator were added to the flask, and the contents of the flask were deoxygenated by sparging ultrapure Argon for 60 minutes. The reaction flask was then sealed, and the reaction was initiated by heating the mixture to 70° C. Polymerization was allowed to proceed for 4 h, and the reaction was quenched by allowing the reaction mixture to cool to room temperature. The resulting polyacrylamide (PAM) polymer was purified by precipitation from large excess of acetone (six times). The recovered polymer was dried under vacuum at 65° C. for 24 h.

### Reduction of PAM RAFT End Group

In a clean, dry beaker, 5.0 g PAM were dissolved in 100.0 mL ethanol, and the resulting mixture was transferred in to 200-mL three-necked round bottom flask equipped with condenser, thermometer, heating mantle, mechanical stirring. Then, 1.0 mL of hydrazine was added dropwise with a syringe. The reaction started immediately along with the disappearance of the pink color of the solution and was allowed to continue for 1 h at room temperature under Argon. After completion of the reaction, the pH of the resulting mixture was adjusted to pH 3.0 using 0.1 M HCl. The solvent was removed with a rotary evaporator. The crude product was dissolved in minimum amount of DMSO followed by precipitation in acetone. After three consecutive purifications, the isolated white precipitate of PAM-SH was dried in a vacuum oven at 65° C. for 24 h.

Thiol-Ene Click Reaction of PAM-SH with Vinyl Sulfone-Functionalized Silica

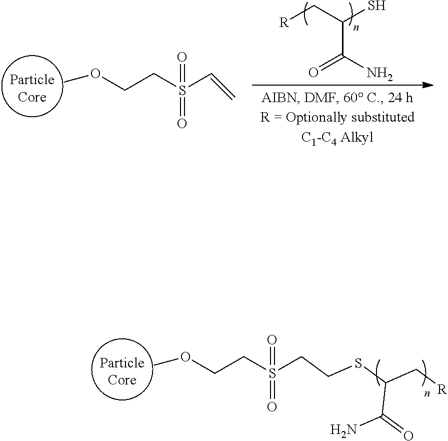

20.0 g BEH silica particles were immersed in 200 mM divinyl sulfone solution in acetonitrile containing 20 mM triphenylphosphine (PPh₃), and the mixture was allowed to react at 25° C. for 12 h. After 12 h, the particles were filtered and washed with acetonitrile (three times). Vinyl sulfone-functionalized silica was recovered and dried at room temperature under vacuum for 12 h prior to use. In a clean, dry beaker, 10.0 g VS-functionalized BEH particles and 5.0 g PAM-SH were dissolved in 200.0 mL DMF followed by sonication for 10 minutes. Then, 50.0 mg AIBN were added, and the resulting solution was transferred to a 250-mL three-necked round bottom flask equipped with condenser, thermometer, heating mantle, mechanical stirring, and Argon purge. The flask was then sealed, and the resulting mixture was sparged with ultrapure Argon for 60 minutes. The reaction was initiated by heating the mixture to 60° C. for 24 h under argon atmosphere. After the reaction reached completion, the reaction was quenched with ice water, and the particles were redispersed in toluene before centrifugation. The purification step was done three more times, and the recovered particles were dried under vacuum at 60° C. for 24 h.

Thiol-Ene Click Reaction of PAM-SH with Methacrylate-Functionalized Silica

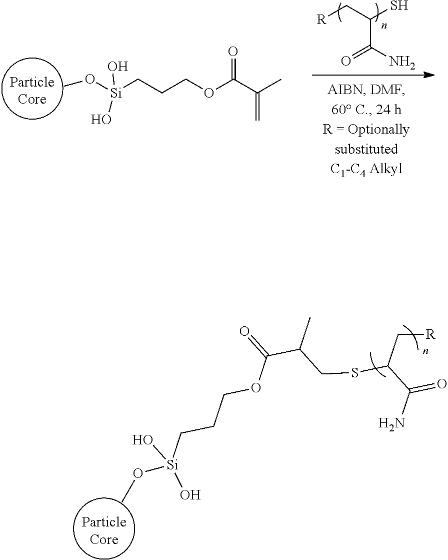

In a 250-mL three-necked round bottom flask equipped with condenser, thermometer, heating mantle, and mechanical stirring, 20.0 g BEH silica particles were dispersed in 100.0 mL toluene. The slurry was subjected to azeotropic distillation for 2 h. Then, 3.2 mg hydroquinone, 0.9 g diethylamine, and 0.9 g methacryloxypropyltrichlorosilane were added to the reaction mixture. The reaction was carried out by refluxing the reaction mixture for 4 h. After the reaction, the bonded particles were recovered via filtration and washed subsequently with toluene, water, and acetone. The washed particles were hydrolyzed with 0.12 M ammonium acetate solution for 3 h at 60° C. and then particles were extensively washed with acetone, water, and methanol. Finally, methacrylate-functionalized BEH particles were dried under vacuum at rt for at least 16 h. In a clean, dry beaker, 10.0 g methacrylate-functionalized BEH particles and 5.0 g PAM-SH were dissolved in 200.0 mL DMF followed by sonication for 10 minutes. Then, 50.0 mg AIBN was added, and the solution was transferred to a 250-mL three-necked round bottom flask equipped with condenser, thermometer, heating mantle, mechanical stirring and Argon purge. The flask was then sealed, and the solution was sparged with ultrapure Argon for 60 minutes. The reaction was initiated by heating the mixture to 60° C. for 24 h under argon atmosphere. After the reaction, the flask was quenched in ice water and the particles were redispersed in toluene and the mixture was centrifuged. The purification step was done three more times, and the particles were recovered and dried under vacuum at 60° C. for 24 h.

### Example 13. Grafting-from Synthesis of HILIC Silica Using RAFT Polymerization

### RAFT Polymerization

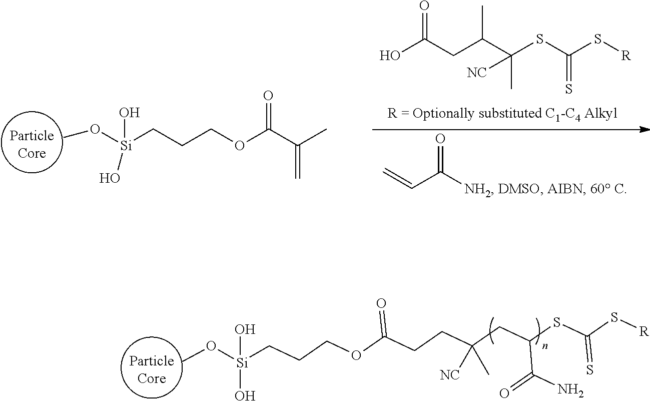

In a 250-mL three-necked round bottom flask equipped with condenser, thermometer, heating mantle, and mechanical stirring, 20.0 g BEH silica particles were dispersed in 100.0 mL toluene. The slurry was subjected to azeotropic distillation for 2 h. Then, 3.2 mg hydroquinone, 0.9 g diethylamine, and 0.9 g methacryloxypropyltrichlorosilane were added to the reaction mixture, which was refluxed for 4 h. After the reaction reached completion, the bonded particles were recovered via filtration and washed subsequently with toluene, water, and acetone. The washed particles were hydrolyzed with 0.12 M ammonium acetate solution for 3 h at 60° C., and then the particles were extensively washed with acetone, water, and methanol. Finally, the obtained methacrylate-functionalized BEH particles were dried under vacuum at room temperature for at least 16 h. In a clean, dry beaker, 10.0 g acrylamide monomer and 5.0 g methacrylate-functionalized silica were dispersed in 70.0 mL dimethyl sulfoxide (DMSO) via sonication, and the resulting mixture was transferred in a 100-mL three-necked round bottom flask equipped with condenser, thermometer, heating mantle, mechanical stirring and Argon purge. Then, 49.1 mg CTA (chain transfer agent) and 12.3 mg 4,4′-azobis(4-cyanovaleric acid) radical initiator were added to the flask. The contents of the flask were deoxygenated by sparging ultrapure Argon for 60 minutes. Upon sealing the reaction flask, the reaction was initiated by heating the mixture to 70° C. Polymerization was allowed to proceed for 4 h, and the reaction was quenched by allowing the reaction mixture to cool to room temperature. The particles were purified by filtration and subsequent washing with water, DMF, and methanol. The recovered polymer was dried under vacuum at 65° C. for 24 h.

Although the disclosure herein has been described with reference to particular examples, it is to be understood that these examples are merely illustrative of the principles and applications of the present disclosure. It is therefore to be understood that numerous modifications may be made to the illustrative examples and that other arrangements may be devised without departing from the spirit and scope of the present invention as defined by the appended claims.

1. A composition comprising a non-porous particle core coated with a hydrophilic polymer with surface-grafted polyionic chains, wherein the polyionic chains have a structure represented by Formula I, II or III: 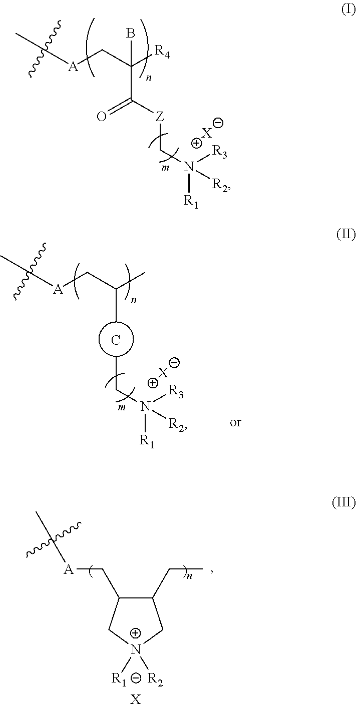
wherein
the squiggly line depicts the point of attachment to surface of the coated polymer particle core;
A is an anchor group selected from —O—, —S—, —C(O)—, —OC(O)—, optionally substituted C₁-C₄ alkyl, C₁-C₄ thioalkyl, amino, C₁-C₄ alkylamine, C₁-C₄ alkylamide, or C₁-C₄ alkyl ester group;
B is H, halogen, or optionally substituted C₁-C₄ alkyl or carbonotrithioate group;
C is aryl or heteroaryl;
n is degree of polymerization ranging from 10-150 repeating monomer units;
Z is NH or O, or Z and one of R₁, R₂, and R₃, together with the atoms to which they are attached, form a C₄-C₆ heterocyclic;
m is from 1-10;
R₁, R₂, and R₃ are each independently a lone pair, H, or optionally substituted C₁-C₄ alkyl, or R₁, and R₂, together with the atoms to which they are attached, form a C₃-C₆ heterocyclic;
R₄ is H, halogen, or methyl; and
X is a counter ion, provided that when one of R₁, R₂, and R₃ in Formula I or II is a lone pair, or R₁ or
R₂ in Formula III is a lone pair, X is absent.

2. The composition of claim 1, wherein the particle core comprises an organic polymer having a polymer backbone that contains C—C covalent bonds, C—O covalent bonds, C—N covalent bonds, O—N covalent bonds, or a combination thereof.

3. (canceled)

4. (canceled)

5. (canceled)

6. The composition of claim 1, wherein is the particle core is coated with an intermediate organic polymer primer layer.

7. The composition of claim 6, wherein the intermediate organic polymer primer layer comprises a hydrophilic polymer copolymerized from the particle surface of a crosslinker.

8. The composition of claim 7, wherein the intermediate organic polymer primer layer comprises ethylene dimethacrylate (EDMA) and a functional monomer bearing epoxy or hydroxyl group.

9. The composition of claim 8, wherein is the intermediate organic polymer primer layer is an EDMA-glycidyl methacrylate (GMA) layer.

10. The composition of claim 1, wherein the hydrophilic coating of the particle core comprises a hydrophilic polymer layer with either random structures or with multiple domains with different chemistries.

11. The composition of claim 1, wherein A is an optionally substituted C₁-C₄ alkyl, C₁-C₄ thioalkyl, or C₁-C₄ alkyl ester group.

12. The composition of claim 11, wherein A is

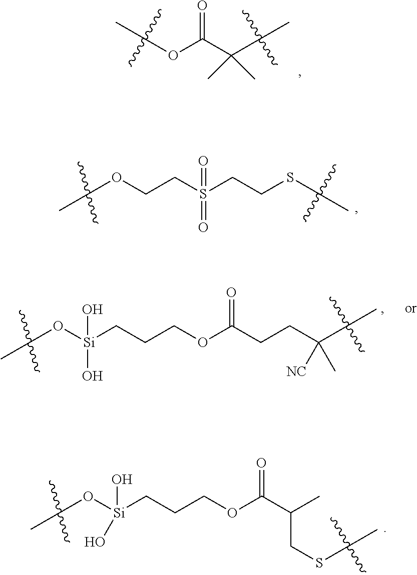

13. The composition of claim 1, wherein B is hydrogen or methyl.

14. (canceled)

15. The composition of claim 1, wherein C is phenyl or C₅-C₆ heteroaryl.

16. The composition of claim 1, wherein the polyionic chains are of Formula (I) or (II), and R₁, R₂, and R₃ are each independently H, methyl, ethyl, propyl, isopropyl, t-butyl, or benzyl.

17. The composition of claim 1, wherein the polyionic chains are of Formula I or II, and one of R₁, R₂, and R₃ is a lone pair.

18. The composition of claim 1, wherein the polyionic chains are of Formula III, and R₁ and R₂ are each independently H, methyl, ethyl, propyl, isopropyl, t-butyl, or benzyl.

19. The composition of claim 1, wherein the polyionic chains are of Formula III, and R₁ or R₂ is a lone pair.

20. The composition of claim 1, wherein the polyionic chains have any one of structures: 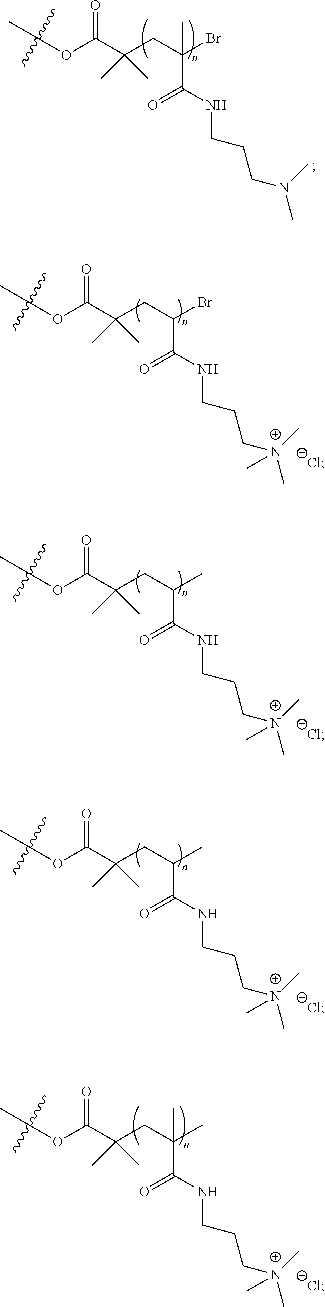 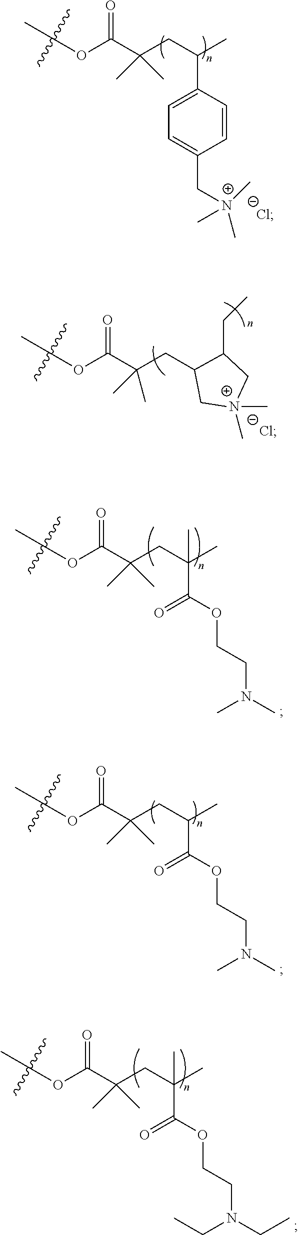 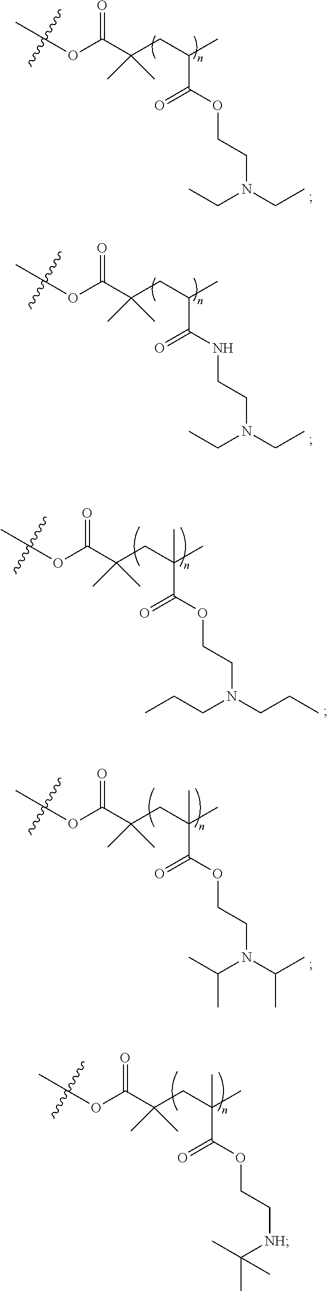 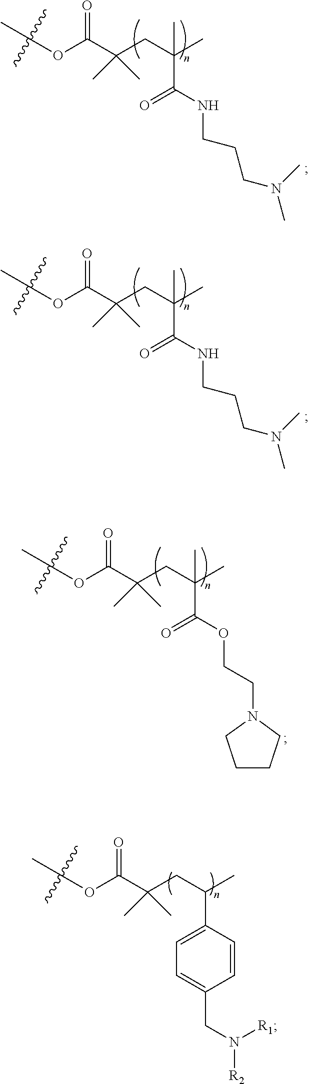 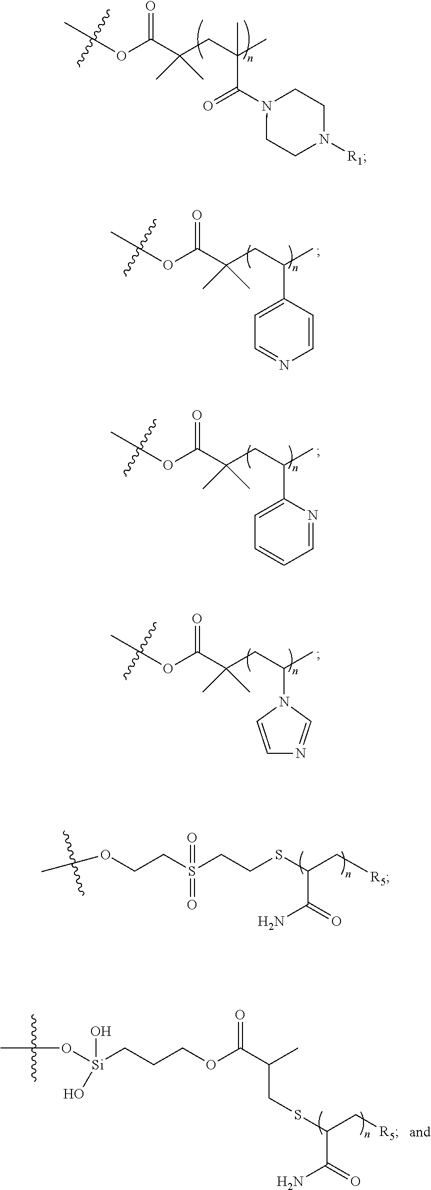 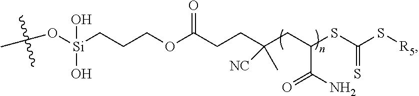
wherein R₅ is optionally substituted C₁-C₄ alkyl.

21. The composition of claim 1, wherein the polyionic chains are strong anion exchangers, weak anion exchangers, or a mixture thereof.

22. (canceled)

23. The composition of claim 1, which has a grafting density between 0.5-15 μmol chains/m².

24. A chromatographic separation device that comprises the composition of claim 1.

25. A chromatographic method comprising: (a) loading a sample onto a chromatographic column comprising the composition of claim 1 and (b) flowing a mobile phase through the column.

26. (canceled)

27. (canceled)
